(19) United States
(12) Patent Application Publication
(10) Pub. No.: US 2013/0177582 A1
(43) Pub. Date: Jul. 11, 2013

(54) PARAPOXVIRUS EXPRESSING THE VP60 MAJOR CAPSID PROTEIN OF THE RABBIT HAEMORRHAGIC DISEASE VIRUS

(71) Applicant: Hanns-Joachim Rziha, Tuebingen (DE)

(72) Inventor: Hanns-Joachim Rziha, Tuebingen (DE)

(73) Assignee: RIEMSER ARZNEIMITTEL AG, Greifswald - Insel Riems (DE)

(21) Appl. No.: 13/643,547

(22) PCT Filed: Apr. 29, 2011
(86) PCT No.: PCT/EP2011/056840

§ 371 Date: Mar. 21, 2013

Related U.S. Application Data
(60) Provisional application No. 61/333,331, filed on May 11, 2010.

(30) Foreign Application Priority Data
Apr. 29, 2010 (EP) 10075174.2
Oct. 8, 2010 (DE) 10 2010 047 920.9

(51) Int. Cl. A61K 39/12; A61K 45/06; C07K 14/08; C12N 7/00

(52) U.S. Cl. CPC A61K 39/12; C12N 7/00; A61K 45/06; C07K 14/08
USPC 424/186.1; 435/235.1; 536/23.72; 530/350

(57) ABSTRACT

The invention describes a therapeutic agent capable of treating and/or preventing rabbit haemorrhagic disease virus infection in rabbits, namely a recombinant parapoxvirus, characterized in that the VP60 major capsid protein from the rabbit haemorrhagic disease virus (RHDV) is expressed from a foreign nucleic acid.

The invention relates to a therapeutic agent capable of treating and/or preventing rabbit haemorrhagic disease virus infection in rabbits, namely a recombinant parapoxvirus, characterized in that the VP60 major capsid protein from the rabbit haemorrhagic disease virus (RHDV) is expressed from a foreign nucleic acid.

### BACKGROUND OF THE INVENTION

Rabbit haemorrhagic disease virus (RHDV) is a highly contagious disease in wild and domestic rabbits, such as of the species Oryctolagus cuniculus, which was first reported in the People's Republic of China in 1984. RHDV subsequently spread throughout Europe during the years 1987 to 1989. Infected rabbits-adults usually die within 48 to 72 hours after infection of necrotizing hepatitis and haemorrhagic syndrome. Morbidity and mortality rates in a population can be as high as 90-100%.

The disease is responsible for significant economic losses in commercial rabbit production, as well as for a high mortality rate in wild rabbits. Commercial rabbit production across the world is an important industry, particularly in Asia and central Europe where small scale rabbit husbandry is well established. The highly contagious and fatal nature of RHDV has had profound economic effects throughout many countries. Additional conservational aspects, such as rabbit population control, in countries such as New Zealand and Australia have led to a significant international effort to understand and control the disease.

Work in many laboratories has led to the characterization of the RHD virus as a calicivirus on the basis of capsid morphology, nucleic acid type and other physical characteristics. The virus particle consists of an un-enveloped icosahedral 35-40 nm diameter capsid, composed primarily of a major 60 kDa polypeptide species. The virus is encoded by a positive sense single strand RNA genome of approximately 7.4 kb and is organized as two open reading frames that code for the major 60 kDa (VP60) capsid protein, a putative protein of 12 kDa, and three non-structural proteins, including an RNA polymerase and a protease. The viral capsid results from the multimerization of approximately 180 copies of a single VP60 protein.

Various efforts have been made to produce vaccines for the treatment and prevention of RHDV infection in rabbit populations, most of which rely on administration of the VP60 capsid protein. VP60 capsid protein has been produced in various expression systems, such as yeast (Boga et al, Journal of General Virology (1997), 78, 2315-2318), Baculovirus/cell culture systems (Laurent et al, Journal of Virology, Oct. 1994, 6794-6798, Marin et al., Virus Research 39 (1995) 119-128) and Pichia pastoris (O. Farnós et al., Antiviral Research 81 (2009) 25-36). Recombinantly produced VP60 protein has been subsequently applied as an effective vaccine. However the production of recombinant protein is associated with various difficulties such as formation of structurally irrelevant protein aggregates, time consuming and complicated protein purification systems and difficulties producing sufficient quality and quantities for commercial production.

Recombinant VP60 has also been produced in potato plants and subsequently applied as a vaccine in leaf extracts (Castanón et al, Journal of Virology, May 1999, p. 4452-4455). Although effective in producing antibodies directed against VP60 in vaccinated rabbits, extract production from plants is comparatively slow compared to virus production in cell culture. Transgenic plants must be grown, harvested and processed, thus limiting output to a scale inappropriate for commercial application. Similarly pea-derived recombinant VP60 can lead to poor yield and thus limited commercial application (Mikschofsky H et al., Plant Biotechnol J. 2009 Aug;7(6):537-549). Similar complications are evident in related attempts to produce the VP60 antigen via expression in plants. Expression in carrots leads to insufficient expression levels for effective vaccination (Mikschofsky, H. et al, 2009, Journal of Agricultural Science 147, 43-49, Mikschofsky, H. et al, 2009, In Vitro Cell.Dev.Biol.Plant 45, 740-749). Expression tobacco plants using the cholera tocxin B as a functional adjuvant also revealed that expression levels of VP60 were sub-optimal, in addition to the further complications associated with production in a plant expression system (Mikschofsky, H. et al, 2009, Plant Sci. doi: 10.1016/j.plantsci.2009.03.010 (Cholera toxin B as a functional adjuvant in tobacco plants).

The use of viruses as RHDV vaccines offers a promising alternative to the administration of recombinant VP60 protein. Vaccinia based vaccines have been applied for RHDV (Bertagnoli et al., Vaccine, Vol. 14, No. 6, pp. 506-510, 1996), although long-term immunity induced by vaccinia virus can result in unsuccessful revaccination or reduced protection against vaccinia-encoded foreign antigens.

At the present time, due to the inability of the RHDV to be cultures in cell culture systems (therefore hindering viral-based vaccine production), present vaccines involve infection and sacrifice of animals in order to extract liver from which the RHDV can be subsequently isolated. This leads to inactivated or dead viral vaccines which are obtained via infection and sacrifice, a practice that has obvious disadvantages in terms of animal protection and financial limitations. RHDV won from infected animals (in the vaccines of the prior art) is subsequently inactivated before application in vaccination. This leads to a significant reduction in activity in comparison to the preferably living parapox-VP60 virus of the present invention. It also requires additional viral inactivation products during its manufacture and often additional adjuvants before application, in order to increase the immune response generated after vaccination.

The Parapoxvirus (PPV) presents a genus of the poxviridae, with the type-species PPV ovis virus (Orf virus, ORFV), which offers several potential advantages for use as a vaccine vector (Rziha et al., Journal of Biotechnology 73 (1999) 235-242). Arguments in favour of an ORFV vaccine vector include the restricted host range (sheep and goats), its tropism being restricted to the skin, the lack of systemic infection, a short-term vector-specific protective immunity and the exceptionally strong stimulation of fast innate cellular immune mechanisms at the site of infection (Büttner, M., and H. J. Rziha. 2002. J. Vet. Med. B Infect. Dis. Vet. Public Health 49:7-16.).

ORFV recombinants have been successfully administered as a vaccine against various virus infections, such as for Pseudorabies virus in pigs (Dory et al., Vaccine 24 (2006) 6256-6263, and van Rooij et al., Vaccine 28 (2010) 1808-1813) or in mice (Fischer et al., Journal of Virology, Sept. 2003, p. 9312-9323) as well as against Borna disease virus in rats (Henkel et al., J. of Virology 2005, p. 314-325).

The ORFV has however not been applied in rabbits or as a vaccine for RHDV. Despite recent advances in vaccines against RHDV, a strong demand remains for novel approaches towards controlling RHDV infection in both wild and commercial rabbit populations.

### SUMMARY OF THE INVENTION

In light of the prior art the technical problem underlying the present invention is to provide an effective therapeutic agent capable of treating and/or preventing rabbit haemorrhagic disease virus infection in rabbits.

This problem is solved by the features of the independent claims. Preferred embodiments of the present invention are provided by the dependent claims.

The invention therefore relates to a recombinant parapoxvirus comprising the VP60 major capsid protein from the rabbit haemorrhagic disease virus (RHDV). The Orf virus offers a restricted host range (sheep and goats) and tropism, and is thus a comparably safe viral vector.

In one embodiment the parapoxvirus of the present invention is characterized in that the VP60 major capsid protein from the rabbit haemorrhagic disease virus (RHDV) is expressed from a foreign nucleic acid sequence. The VP60 protein of the RHDV expressed from the recombinant Parapoxvirus acts to induce an antigen-specific immunity in treated animals which provides an effective protection from RHDV infection.

In one embodiment the parapoxvirus of the present invention is characterized in that the parapoxvirus is an Orf virus (ORFV), preferably the ORFV strain D1701, more preferably D1701-V-VP60n.

In one embodiment the parapoxvirus of the present invention is characterized in that the foreign nucleic acid sequence is a DNA sequence integrated into the parapoxvirus genome.

In one embodiment the parapoxvirus of the present invention is characterized in that the VP60 major capsid protein is encoded by a DNA sequence comprising the sequence according to SEQ ID No. 1 or SEQ ID No. 2.

In one embodiment the parapoxvirus of the present invention is characterized in that the VP60 major capsid protein comprises the amino acid sequence according to SEQ ID No. 3.

A further aspect of the present invention relates to the parapoxvirus as described herein for use as a medicament.

A further aspect of the present invention relates to the parapoxvirus as described herein for use as a vaccine in the therapeutic and/or prophylactic treatment of RHDV infection in a subject.

A further aspect of the present invention relates to a pharmaceutical composition comprising the parapoxvirus according to the present invention, a nucleic acid molecule encoding the VP60 protein according to SEQ ID No. 1 or SEQ ID No. 2 and/or the VP60 protein comprising the sequence according to SEQ ID No. 3 and a pharmaceutically acceptable carrier.

A further aspect of the present invention relates to use of the parapoxvirus as described herein as a vaccine in the therapeutic and/or prophylactic treatment of RHDV infection in a subject.

A further aspect of the present invention relates to a vaccine comprising the parapoxvirus as described herein, whereby the parapoxvirus is preferably alive. Living attenuated viruses are preferred. In one embodiment the vaccine as described herein can be characterised in that additional agents are present for enhanced vaccine function, such as adjuvants or immune stimulators. In a further embodiment the vaccine as described herein can be characterised in that the vaccine is a combination vaccine, comprising of vaccine-agents for additional diseases.

A further aspect of the present invention relates to a method of therapeutic and/or prophylactic treatment of RHDV infection in rabbits, characterised in that a therapeutically effective amount of the parapoxvirus according to the invention, the pharmaceutical composition of the invention or the vaccine as described herein is administered to a subject.

A further aspect of the present invention relates to a nucleic acid molecule encoding the rabbit haemorrhagic disease virus (RHDV) protein VP60 comprising the sequence according to SEQ ID No. 1 or SEQ ID No. 2. In one embodiment the nucleic acid molecule coding for VP60 is intended for use in the production of a recombinant parapoxvirus as described herein.

A further aspect of the present invention relates to a VP60 major capsid protein of the rabbit haemorrhagic disease virus (RHDV) comprising the sequence according to SEQ ID No. 3.

A further aspect of the present invention relates to a use of the nucleic acid molecule coding for VP60 and/or the VP60 major capsid protein in the manufacture of a medicament, preferably a vaccine, for the therapeutic and/or prophylactic treatment of RHDV infection in rabbits.

The subject of the present invention is intended to a rabbit. “Rabbit” is to be understood as mammals in the family Leporidae of the order Lagomorpha. Wild and domesticated rabbits are intended subjects of the present invention. There are eight different genera in the family classified as rabbits, including the European rabbit (Oryctolagus cuniculus) and cottontail rabbits (genus Sylvilagus), in addition to many other species of rabbit, included are also hares.

### DETAILED DESCRIPTION OF THE INVENTION

In one aspect, the present invention relates to a recombinant parapoxvirus, containing a

DNA sequence coding for the RHDV VP60 major capsid protein. According to the present invention, the recombinant parapoxvirus expresses gene products of the VP60 gene. Specific open reading frame(s) of VP60 are inserted into the parapoxvirus vector, and the resulting recombinant virus is used to immunize an animal. Expression of VP60 gene product results in an immune response in the treated animal targeted specifically against VP60. Thus, the recombinant virus of the present invention may be used in an immunological composition or vaccine to provide a means to induce an immune response which may, but need not be, protective. Said immune response may be therapeutic or prophylactic in its effect.

The combination of the VP60 with parapox virus enables VP60-containing virus to be produced in cell culture, something that was until now not successfully achieved. The present invention therefore enables production of VP60-vaccine without sacrifice of infected animals, as is currently carried out in the prior art. The parapoxvirus described herein is propagated effectively in Vero cells (Monkey kidney cell line).

The VP60 gene is expressed under control of an early parapoxvirus-promoter once integrated in the parapox genome. The VP60 gene product is therefore expressed after infection of the cells by the parapox virus, so that the VP60 gene is both transcribed and translated whilst in the host cells. However, this expression does not lead to the creation of infectious virus.

The active expression of recombinantly integrated gene products after infection represents a general principle of the poxviruses and is dependent on living vaccine. The recombinant virus of the present invention can however be applied as either living or dead virus, considering that the VP60 protein is expressed and leads to VP60 antigen production before application. Production of antibodies in host cells in response to the VP60 antigen occurs in response to application of either live or dead virus. In a preferred embodiment the recombinant virus of the present invention is applied to subjects as a living vaccine, as VP60 expression continues after infection.

A significant and unexpected advantage of the present invention is that subjects infected (treated or vaccinated) with the virus vaccine of the present invention do not produce virus particles, which cannot then be released from the treated subject. This represents a significant advantage for various reasons. Firstly, infection (vaccination) is limited to those subjects treated initially, without allowing uncontrolled propagation of the virus. The virus can therefore also not spread to other hosts, which is a potential concern despite the limited host range of the parapox virus. Secondly, because the virus is recombinant in nature (genetically modified) there is significant emphasis made by both the general public and by agricultural regulatory bodies that genetically modified products are not able to spread or be released into the environment. The fact that the recombinant viral vaccine of the present invention is not released from infected subjects represents therefore a surprising and advantageous aspect to the invention, which could not have been predicted in light of the prior art.

The present invention relates to all parapox viruses as vectors for the recombinant VP60 gene. In a preferred embodiment the parapoxvirus of the present invention is characterised in that the Parapoxvirus is an Orf virus (ORFV), preferably the ORFV-strain D1701, more preferably the strain D1701-V-VP60n. Derivatives of the ORFV-strain D1701 are intended to fall within the scope of the invention. The strain D1701 represents a highly attenuated virus originally isolated from Sheep. After serial cell culture passages the resulting avirulent D1701 strain was successfully used in the development of a live vaccine (Mayr, A. et al, 1981, Tbl. Vet. Med. B. 28, 535-552). The D1701 strain has been further characterised and examined for its properties as a vaccine vector (Cottone, R. et al, 1998, Virus Research 56, 53-67, Rziha, H.-J. et al, 2000, Journal of Biotechnology 83, 137-145, EP 0 886 679 B1). The strains described in Mayr et al (1981), Cottone et al (1998), Rziha et al (2000) and in EP 0 886 679 B1 are intended as preferred parapox vectors for the VP60 gene as described herein. These strains are highly attenuated, apathogenic and unable to reproduce in rabbits.

A further advantage of the recombinant parapoxvirus of the present invention is the immune stimulation effect that occurs during treatment of subjects. The parapoxvirus enhances the immune stimulatory effect, so that the virus vector itself acts a adjuvant for the VP-60 application. This unique and inventive combination of the parapox virus with the VP60 gene results in a synergistic effect, whereby the immune stimulatory properties of the parapoxvirus combine in a synergistic manner with VP60 antigen, which also induces an immune response (namely an antigen specific immune response), thereby providing a strong immune response towards the VP60 antigen. This combination of factors ultimately results in production of an effective vaccine. Until now, the approaches as described in the prior art that attempted to administer VP60 in various forms had never led to a sufficient immune response in order to provide effective protection against the virus. The unique combination of parapoxvirus and VP60 does however provide an effective solution to the long-felt need for a RHDV vaccine. As has been shown in the prior art, application of either parapoxvirus or VP60 antigen alone has no particular effect. The combination leads to surprising effectiveness, whereby each of the virus and VP60 produce an effect together that is greater than the sum of their parts, generating an immune response that provides lasting protection against the RHDV.

The administration procedure for recombinant virus of the present invention or expression product thereof, compositions of the invention such as immunological, antigenic or vaccine compositions or therapeutic compositions, can be administered via parenteral routes, such as intradermal, intramuscular or subcutaneous application methods. Such an administration enables a systemic immune response, or humoral or cell-mediated immune responses. The vaccine according to the present invention can also be incorporated in animal feed, enabling simple and effective immunisation of large populations.

The preferred methods of application relate to either sub-cutaneous application or intramuscular application, preferably by injection.

More generally, the inventive VP60 parapoxvirus recombinant, antigenic, immunological or vaccine parapoxvirus-VP60 (D1701-V-VP60n) compositions or therapeutic compositions can be prepared in accordance with standard techniques well known to those skilled in the pharmaceutical or veterinary art.

A therapeutically effective amount of such compositions can be administered in dosages and by techniques well known to those skilled in the medical or veterinary arts taking into consideration such factors as the age, sex, weight, species and condition of the particular subject, and the route of administration. The compositions can be administered alone, or can be co-administered or sequentially administered with compositions, e.g., with “other” immunological, antigenic or vaccine or therapeutic compositions thereby providing multivalent or “cocktail” or combination compositions of the invention and methods employing them. Again, the ingredients and manner (sequential or co-administration) of administration, as well as dosages can be determined taking into consideration such factors as the age, sex, weight, species and condition of the particular subject, and, the route of administration.

Examples of compositions of the invention include liquid preparations for orifice, e.g., oral, nasal, anal, vaginal, peroral, intragastric, etc., administration such as suspensions, syrups or elixirs; and, preparations for parenteral, subcutaneous, intradermal, intramuscular or intravenous administration (e.g., injectable administration) such as sterile suspensions or emulsions.

In such compositions the recombinant parapoxvirus may be in admixture with a pharmaceutically acceptable carrier, diluent, or excipient such as sterile water, physiological saline, glucose or the like. The compositions can also be lyophilized. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired.

TABLE 1
Sequences of the present invention

| | DNA/ | SEQ ID | |
|---|---|---|---|
| Description | protein | NO. | Sequence (5′-3′) |
| VP60 gene | DNA | SEQ ID | ATGGAGGGCAAAGCCCGTGCAGCGCCGCAAGGCGAA |
| sequence | | NO. 1 | GCAGCGGGCACTGCCACCACAGCATCAGTTCCCGGAA |
| | | | CCACGACTGATGGCATGGATCCTGGCGTTGTGGCCAC |
| | | | TACCAGCGTGATCACTGCAGAAAATTCATCCGCATCGA |
| | | | TTGCAACAGCAGGGATTGGCGGACCACCCCAACAGGT |
| | | | GGACCAACAAGAGACATGGAGAACAAATTTTTATTACA |
| | | | ATGATGTTTTCACTTGGTCAGTCGCGGACGCCCCTGG |
| | | | CAGCATACTTTACACCGTCCAACATTCTCCACAAAACA |
| | | | ACCCATTCACAGCCGTGCTGAGCCAGATGTACGCTGG |
| | | | CTGGGCTGGTGGCATGCAGTTTCGCTTCATAGTTGCC |
| | | | GGGTCAGGTGTGTTTGGTGGGCGACTGGTCGCGGCT |
| | | | GTGATACCACCAGGCATCGAGATTGGACCAGGGTTGG |
| | | | AGGTTAGGCAGTTTCCTCATGTTGTCATCGATGCCCGT |
| | | | TCACTCGAACCTGTTACCATCACCATGCCAGACTTGCG |
| | | | TCCCAACATGTACCATCCAACTGGTGACCCTGGCCTT |
| | | | GTTCCCACACTAGTCCTTAGTGTTTACAACAACCTTAT |
| | | | CAACCCGTTTGGTGGGTCCACTAGTGCAATCCAGGTG |
| | | | ACAGTGGAAACAAGGCCAAGTGAAGACTTTGAGTTCG |
| | | | TGATGATTCGAGCCCCCTCCAGCAAGACTGTTGACTC |
| | | | AATTTCGCCCGCAGGCCTCCTCACGACCCCAGTCCTT |
| | | | ACTGGGGTTGGCAATGACAACAGGTGGAACGGCCAAA |
| | | | TAGTGGGACTGCAACCAGTACCTGGAGGGTTTTCTAC |
| | | | GTGCAACAGGCATTGGAACTTGAATGGCAGCACATAT |
| | | | GGCTGGTCAAGCCCTCGGTTTGCCGACATTGACCATC |
| | | | GAAGAGGCAGTGCAAGTTACTCTGGGAGCAACGCAAC |
| | | | CAACGTGCTTCAGTTTTGGTATGCCAATGCTGGGTCTG |
| | | | CAATCGACAACCCCATTTCCCAGGTTGCACCAGACGG |
| | | | CTTTCCTGACATGTCGTTCGTGCCCTTTAACGGCCCCG |
| | | | GCATTCCAGCTGCGGGGTGGGTTGGATTTGGTGCAAT |
| | | | CTGGAACAGTAACAGCGGTGCCCCCAACGTCACGACT |
| | | | GTGCAGGCCTATGAGTTAGGTTTTGCCACTGGGGCAC |
| | | | CAGGCAACCTCCAGTCCACCACCAACACTTCAGGTGC |
| | | | ACAGACTGTCGCCAAGTCCATTTATGCCGTGGTGACT |
| | | | GGCACAGCCCAAAACCCAGCCGGATTGTTTGTGATGG |
| | | | CCTCGGGTATTATCTCCACCCCAAATGCCAGCGCCAT |
| | | | CACATACACGCCTCAACCAGACAGAATTGTAACCACAC |
| | | | CCGGCACTCCTGCCGCTGCACCTGTGGGTAAGAACAC |
| | | | ACCCATCATGTTTGCGTCTGTCGTCAGGCGCACCGGT |
| | | | GACGTCAACGCCACAGCTGGGTCAGCCAACGGGACC |
| | | | CAGTACGGCACAGGCTCCCAACCACTGCCAGTGACAA |
| | | | TTGGACTTTCGCTCAACAACTACTCGTCAGCACTTATG |
| | | | CCCGGACAGTTTTTCGTTTGGCAGTTAACCTTTGCATC |
| | | | TGGTTTCATGGAGATAGGTTTAAGTGTGGACGGGTATT |
| | | | TTTATGCAGGAACAGGGGCCTCAACCACACTCATTGA |
| | | | CCTGACTGAACTCATTGACGTACGCCCTGTGGGACCC |
| | | | AGGCCGTCCAAAAGCACACTCGTGTTCAACCTGGGGG |
| | | | GCACAGCCAATGGCTTTTCTTATGTCTGA |
| VP60 gene | DNA | SEQ ID | GTGAAGCTTATGGAGGGCAAAGCCCGTGCAGCGCCG |
| sequence | | NO. 2 | CAAGGCGAAGCAGCGGGCACTGCCACCACAGCATCA |
| (optimised; | | | GTTCCCGGAACCACGACTGATGGCATGGATCCTGGCG |
| VP60-new) | | | TTGTGGCCACTACCAGCGTGATCACTGCAGAAAATTCA |
| | | | TCCGCATCGATTGCAACAGCAGGGATTGGCGGACCAC |
| | | | CCCAACAGGTGGACCAACAAGAGACATGGAGAACAAA |
| | | | TTTCTACTACAATGATGTTTTCACTTGGTCAGTCGCGG |
| | | | ACGCCCCTGGCAGCATACTTTACACCGTCCAACATTCT |
| | | | CCACAAAACAACCCATTCACAGCCGTGCTGAGCCAGA |
| | | | TGTACGCTGGCTGGGCTGGTGGCATGCAGTTTCGCTT |
| | | | CATAGTTGCCGGGTCAGGTGTGTTTGGTGGGCGACTG |
| | | | GTCGCGGCTGTGATACCACCAGGCATCGAGATTGGAC |
| | | | CAGGGTTGGAGGTTAGGCAGTTTCCTCATGTTGTCATC |
| | | | GATGCCCGTTCACTCGAACCTGTTACCATCACCATGCC |
| | | | AGACTTGCGTCCCAACATGTACCATCCAACTGGTGAC |
| | | | CCTGGCCTTGTTCCCACACTAGTCCTTAGTGTTTACAA |
| | | | CAACCTTATCAACCCGTTTGGTGGGTCCACTAGTGCAA |
| | | | TCCAGGTGACAGTGGAAACAAGGCCAAGTGAAGACTT |
| | | | TGAGTTCGTGATGATTCGAGCCCCCTCCAGCAAGACT |
| | | | GTTGACTCAATTTCGCCCGCAGGCCTCCTCACGACCC |
| | | | CAGTCCTTACTGGGGTTGGCAATGACAACAGGTGGAA |
| | | | CGGCCAAATAGTGGGACTGCAACCAGTACCTGGAGGG |
| | | | TTTTCTACGTGCAACAGGCATTGGAACTTGAATGGCAG |
| | | | CACATATGGCTGGTCAAGCCCTCGGTTTGCCGACATT |
| | | | GACCATCGAAGAGGCAGTGCAAGTTACTCTGGGAGCA |
| | | | ACGCAACCAACGTGCTTCAGTTTTGGTATGCCAATGCT |
| | | | GGGTCTGCAATCGACAACCCCATTTCCCAGGTTGCAC |
| | | | CAGACGGCTTTCCTGACATGTCGTTCGTGCCCTTTAAC |
| | | | GGCCCCGGCATTCCAGCTGCGGGGTGGGTTGGATTT |
| | | | GGTGCAATCTGGAACAGTAACAGCGGTGCCCCCAACG |
| | | | TCACGACTGTGCAGGCCTATGAGTTAGGTTTTGCCACT |
| | | | GGGGCACCAGGCAACCTCCAGTCCACCACCAACACTT |
| | | | CAGGTGCACAGACTGTCGCCAAGTCCATTTATGCCGT |
| | | | GGTGACTGGCACAGCCCAAAACCCAGCCGGATTGTTT |
| | | | GTGATGGCCTCGGGTATTATCTCCACCCCAAATGCCA |
| | | | GCGCCATCACATACACGCCTCAACCAGACAGAATTGT |
| | | | AACCACACCCGGCACTCCTGCCGCTGCACCTGTGGGT |
| | | | AAGAACACACCCATCATGTTTGCGTCTGTCGTCAGGC |
| | | | GCACCGGTGACGTCAACGCCACAGCTGGGTCAGCCA |
| | | | ACGGGACCCAGTACGGCACAGGCTCCCAACCACTGC |
| | | | CAGTGACAATTGGACTTTCGCTCAACAACTACTCGTCA |
| | | | GCACTTATGCCCGGACAGTTCTTCGTTTGGCAGTTAAC |
| | | | CTTTGCATCTGGTTTCATGGAGATAGGTTTAAGTGTGG |
| | | | ACGGGTATTTCTACGCAGGAACAGGGGCCTCAACCAC |
| | | | ACTCATTGACCTGACTGAACTCATTGACGTACGCCCTG |
| | | | TGGGACCCAGGCCGTCCAAAAGCACACTCGTGTTCAA |
| | | | CCTGGGGGGCACAGCCAATGGCTTTTCTTATGTCT- |
| | | | GATGATTTTTATGAATTCATC |
| VP60 | Protein | SEQ ID | MEGKARAAPQGEAAGTATTASVPGTTTDGMDPGVVATT |
| protein | | NO. 3 | SVITAENSSASIATAGIGGPPQQVDQQETWRTNFYYNDV |
| sequence | | | FTWSVADAPGSILYTVQHSPQNNPFTAVLSQMYAGWAG |
| | | | GMQFRFIVAGSGVFGGRLVAAVIPPGIEIGPGLEVRQFPH |
| | | | VVIDARSLEPVTITMPDLRPNMYHPTGDPGLVPTLVLSVY |
| | | | NNLINPFGGSTSAIQVTVETRPSEDFEFVMIRAPSSKTVD |
| | | | SISPAGLLTTPVLTGVGNDNRWNGQIVGLQPVPGGFSTC |
| | | | NRHWNLNGSTYGWSSPRFADIDHRRGSASYSGSNATN |
| | | | VLQFWYANAGSAIDNPISQVAPDGFPDMSFVPFNGPGIP |
| | | | AAGWVGFGAIWNSNSGAPNVTTVQAYELGFATGAPGNL |
| | | | QSTTNTSGAQTVAKSIYAVVTGTAQNPAGLFVMASGIIST |
| | | | PNASAITYTPQPDRIVTTPGTPAAAPVGKNTPIMFASVVR |
| | | | RTGDVNATAGSANGTQYGTGSQPLPVTIGLSLNNYSSAL |
| | | | MPGQFFVWQLTFASGFMEIGLSVDGYFYAGTGAST- |
| | | | TLIDLTELIDVRPVGPRPSKSTLVFNLGGTANGFSYV |

### FIGURES AND LEGENDS

Short description of the figures:

FIG. 1: Plasmid pMK-RQ Schematic, synthesized by and obtained from Mr. Gene company.

FIG. 2: Nucleotide sequence of the synthetically produced gene VP60 (SEQ ID No. 2)

FIG. 3: Recombinant virus selection via PCR

FIG. 4: Immunostaining of infected cells for VP60 expression

FIG. 5: Integration of the VP60 gene into the D1701-V genome

FIG. 6: Analysis of VP60 transcription in recombinant viruses

FIG. 7: Analysis of VP60 protein expression in recombinant viruses

FIG. 8: Immunofluorescence analysis

FIG. 9: Animal experiments investigating the immunogenicity of the ORF-RHDV-VP60 recombinant virus (D1701-VP60)

FIG. 10: Animal experiments investigating treatment dose

FIG. 11: Cytokine pattern response in mouse serum after treatment with different viral preparations

FIG. 12: Cytokine and Chemokine expression in Serum of Balb/C Mice after intraperitoneal application

### DETAILED DESCRIPTION OF THE FIGURES

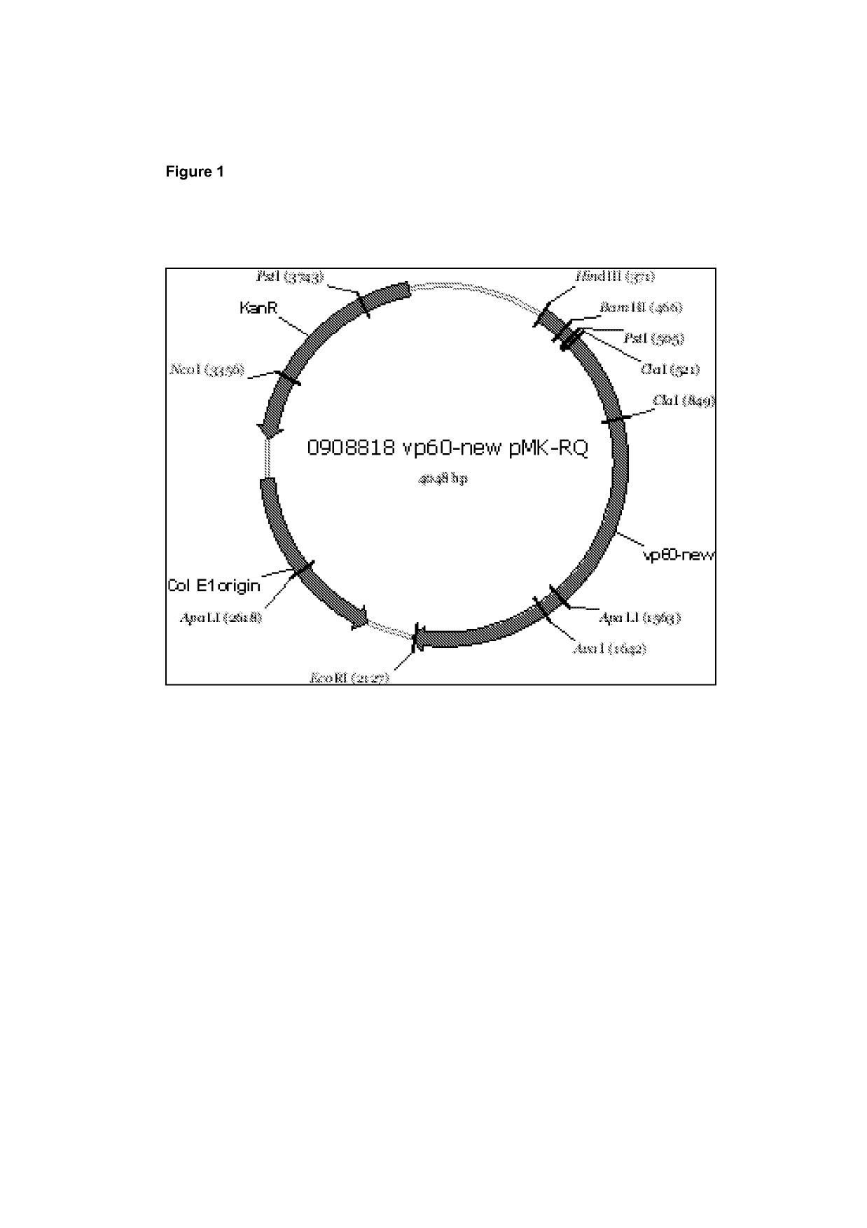

FIG. 1: pMK-RQ Schematic. A schematic representation of the pMK-RQ vector, which contains the VP60 gene is provided. Synthetic VP60 gene, ordered from Mr. Gene (Regensburg, FRG), and obtained as plasmid vp60-new pMK-RQ. Relevant restriction enzyme cleavage sites and the sequence positions (nucleotide numbers) are indicated. The open reading frame of VP60-new is flanked by the HindIII (# 371) and EcoRl site (# 2127), which have been used for cloning the gene into the transfer plasmid pdV-Rec1. Silent mutations were introduced into the VP60 gene to remove poxviral transcript stop motifs T5NT (where N represents any nucleotide). Open reading frames and restriction enzyme sites are shown.

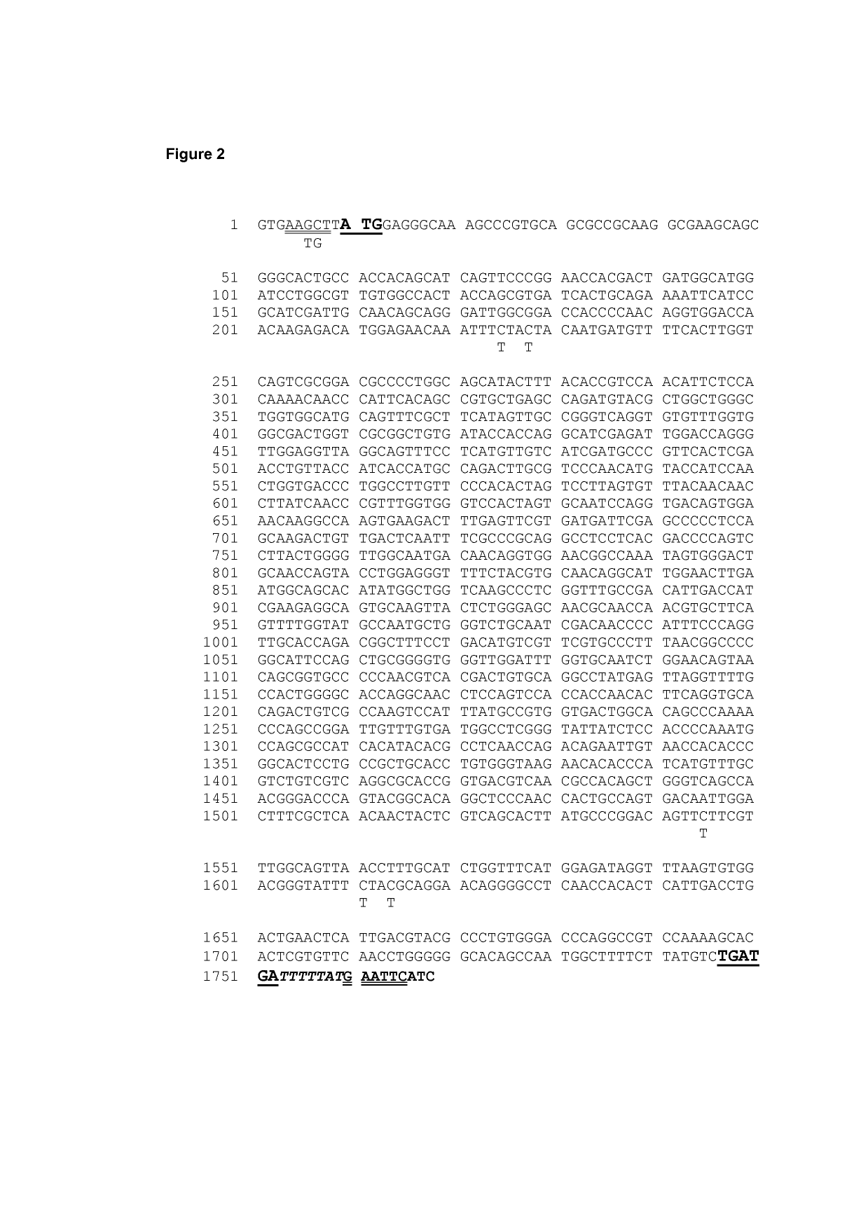

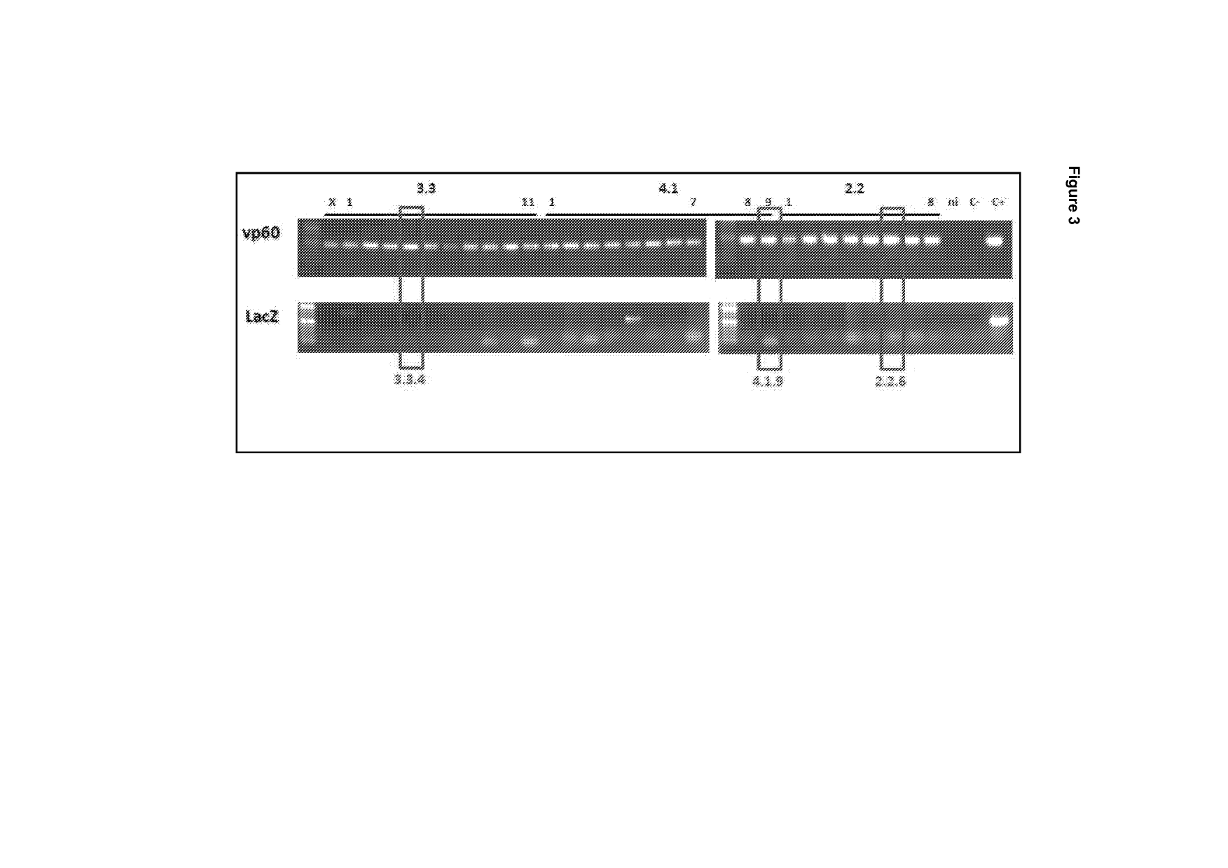

FIG. 2: Nucleotide sequence of the synthetically produced gene VP60 (SEQ ID No. 2). The new nucleotide sequence of the VP60 gene is provided. In those regions were changes have been made in comparison the VP-60 gene sequence known in the art, the previous base has been inserted in the line below the new sequence. All base changes introduced in the open reading frame of VP60 were silent and did not alter the amino acid sequences. Nucleotides 6 and 7 have been changed from T→G and from G→C in order to obtain the HindIII cloning site AAGCTT (double underlined). The translational start codon ATG at position 10 is shown enlarged in bold. Nucleotides # 225 and 228 have been changed from T→C, respectively, to remove the poxviral early transcription stop motif TTTTTAT (T5NT), as changes from T→C have been also introduced at positions 1545, 1611 and 1614, respectively. Directly behind the original translation stop codon TGA (position 1747-1749) a second stop motif was added followed by the introduction of a new T5NT motif needed for ORFV early transcription. At the end of the gene sequence a new EcoRl cloning site (GAATTC) was introduced.

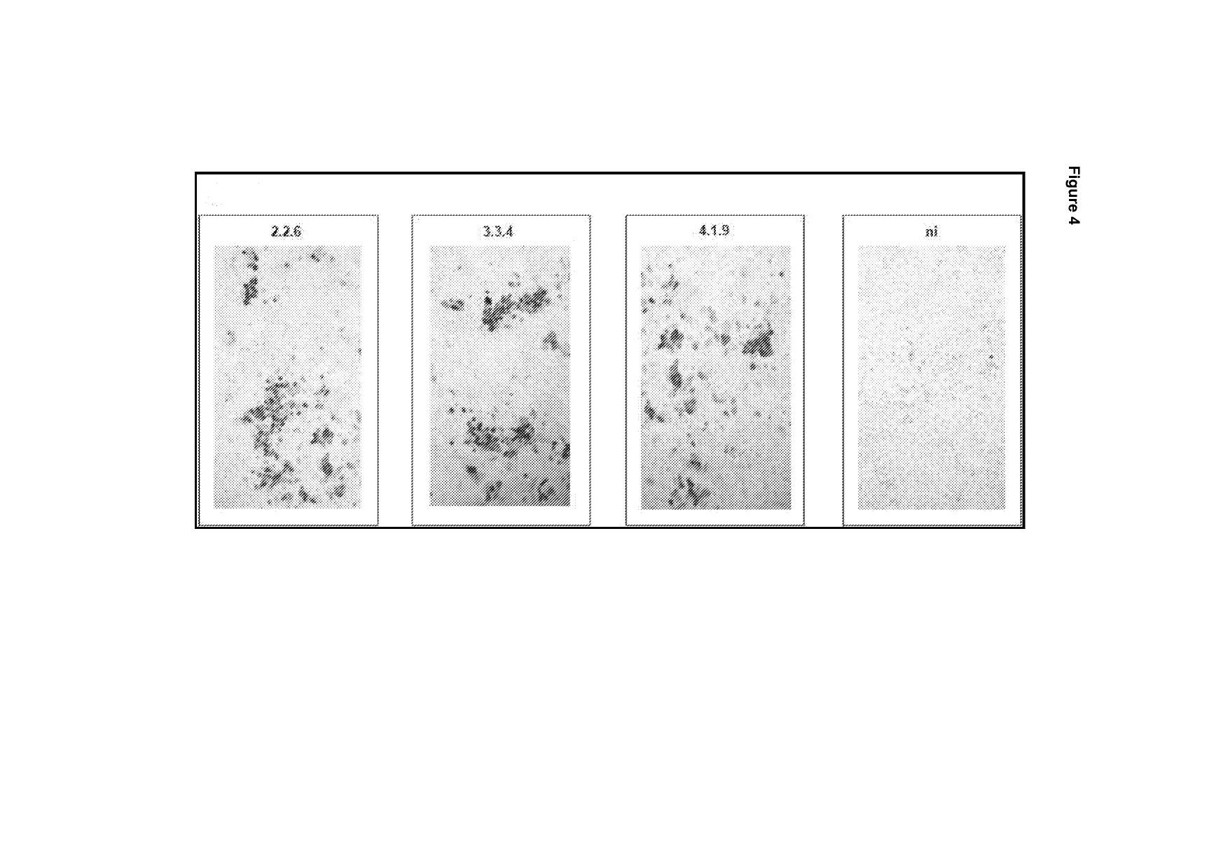

FIG. 3: Recombinant virus selection via PCR. 29 individual plaque isolates were tested via PCR for the presence of the VP60 gene (upper panel). The absence of the LacZ gene was also tested in order to exclude plaques that contained contaminating parental D1701-VrV virus (lower panel). The PCR analysis demonstrates that multiple individual plaques contain the VP60 gene but do not contain the contaminating parental LacZ gene. As controls the following samples were used: ni represents uninfected cells, C− represents a water control, C+ represents 50 ng of pdV-VP60n plasmid DNA (for VP60) or 50 ng of a LacZ gene containing plasmid (for LacZ).

FIG. 4: Immunostaining of infected cells for VP60 expression. Vero cells were fixed 48 hours after infection with the three recombinant plaque isolates (3.3.4, 4.1.9 and 2.2.6) and were subsequently stained with a VP60 specific antiserum (provided by Dr. B. Szewczyk, University of Gdansk, Poland).

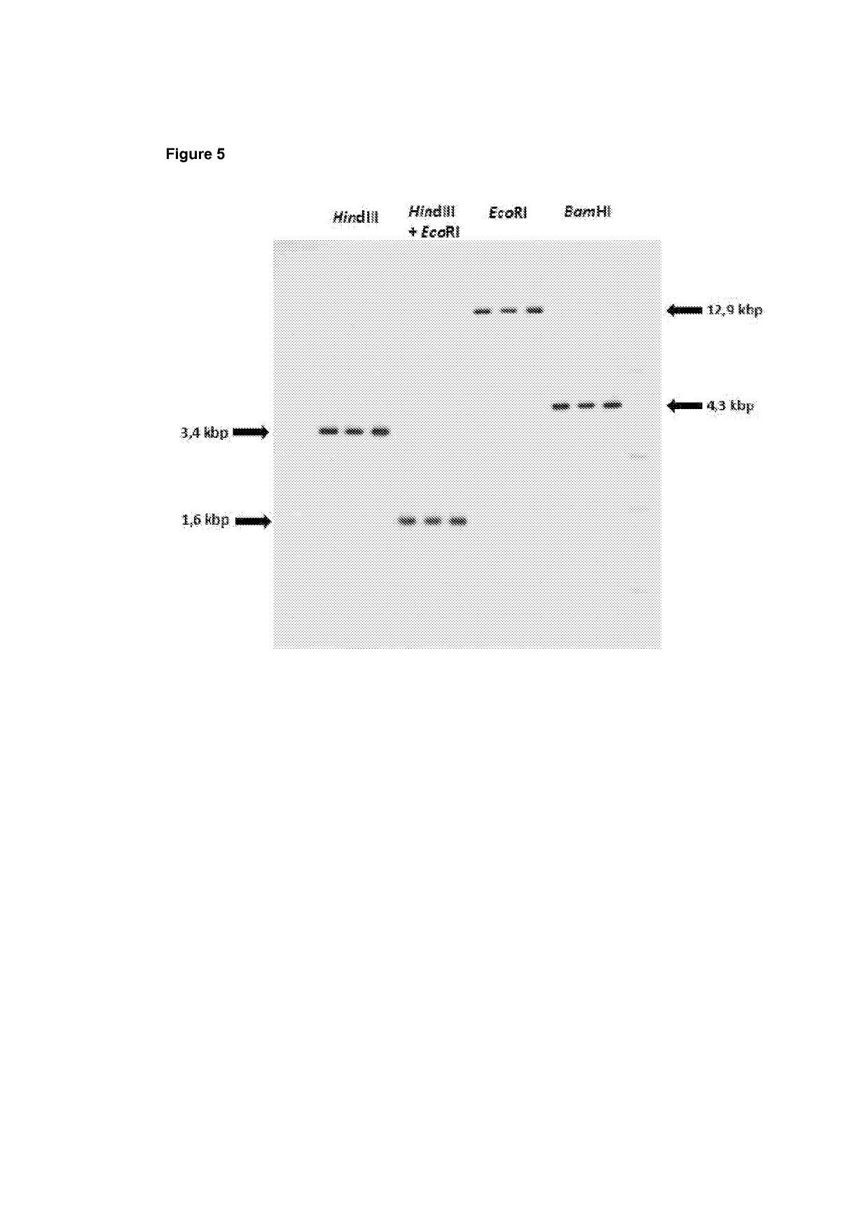

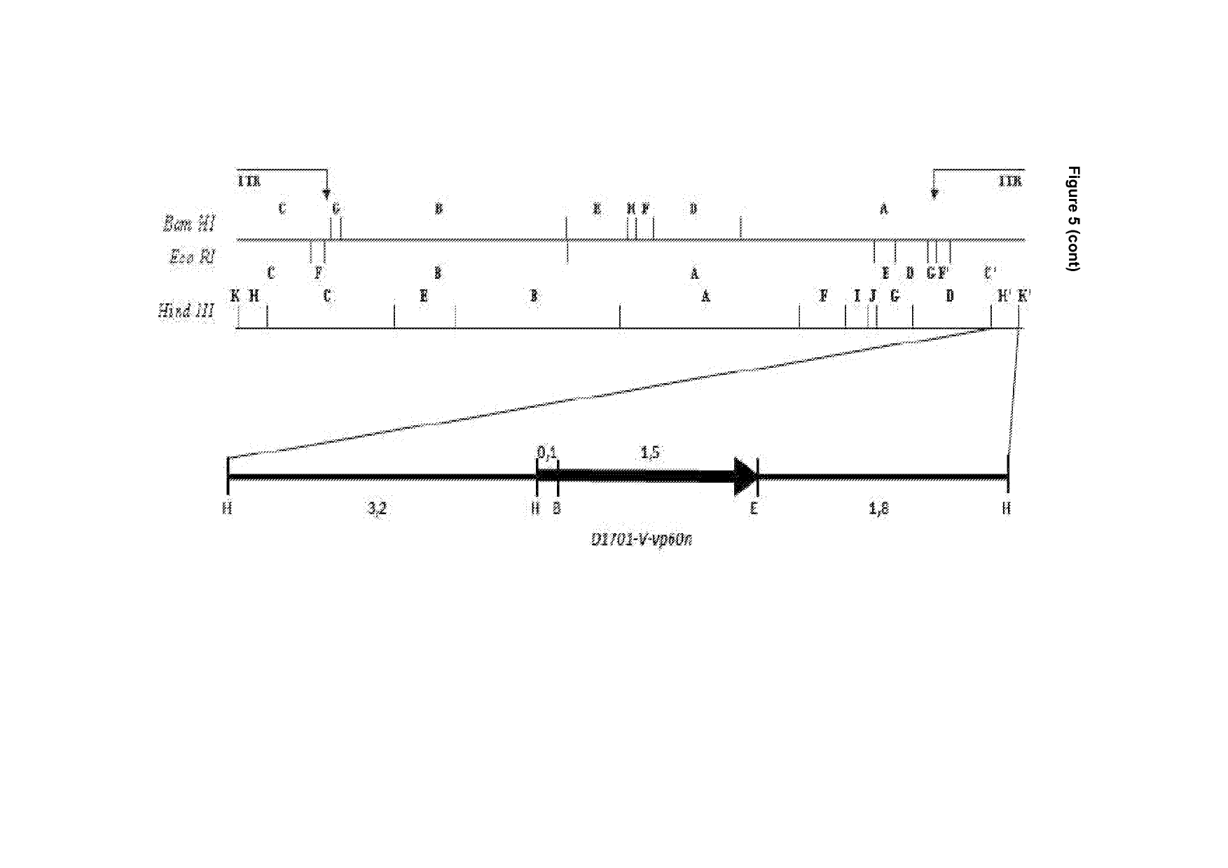

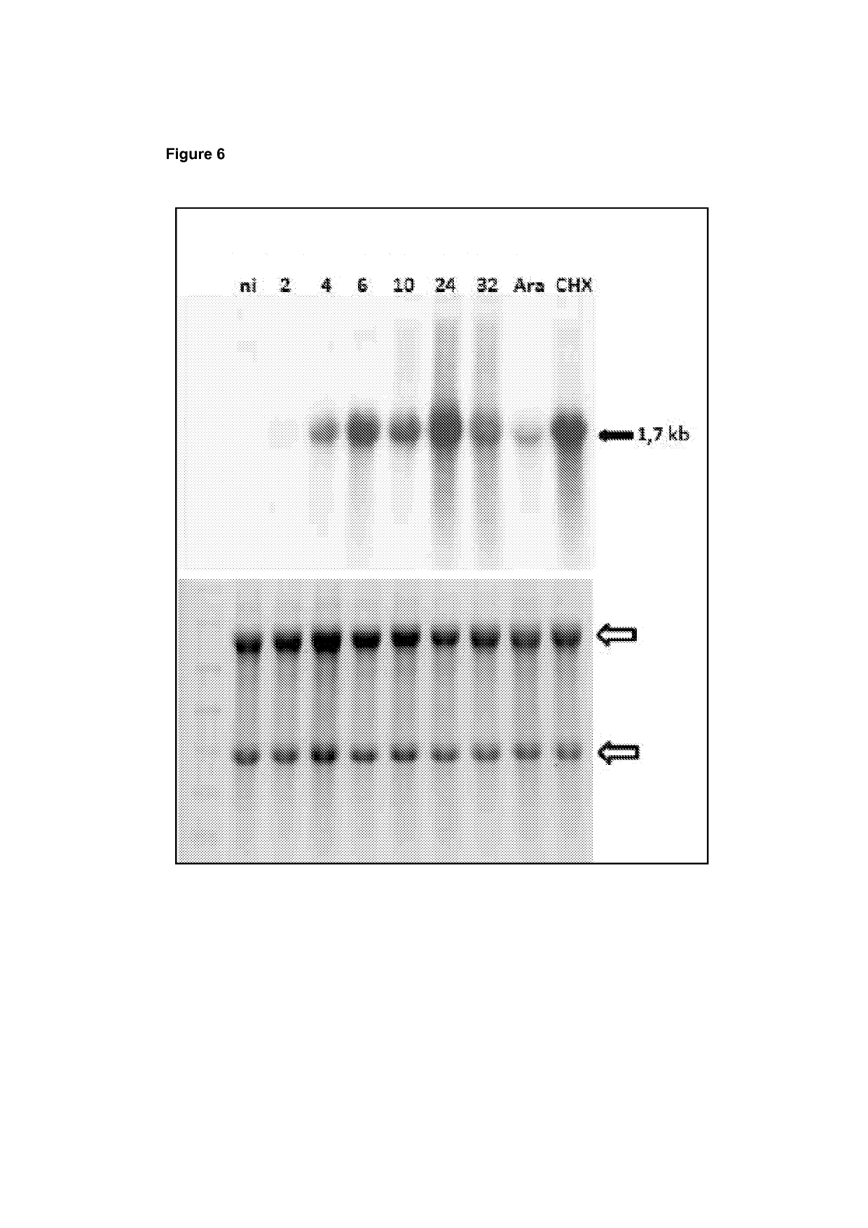

FIG. 5: Integration of the VP60 gene into the D1701-V genome. DNA from the three plaque isolates were digested with restriction enzymes (as shown in the figure) and subsequently probed with a radioactive VP60-specific probe. The hybridisation produced both the expected 3.4 kbp and 1.7 kbp DNA fragments from the D1701-Vvp60 recombinants after Hind III or Hind III plus Eco R1 restriction digestion. The lower half of the figure shows enlarged a schematic representation of the 5.5 kbp HindIII DNA fragment that contains the inserted VP60 gene of RHDV (arrow).

FIG. 6: Analysis of VP60 transcription by northern blot hybridization. Total RNA was isolated from cells 2, 4, 6, 10, 24, and 32 hours after infection as well as after 24 hours in the presence of either cytosine arabinoside (AraC) or cyclohexamide (CHX). Equal amounts of RNA (6 μg) were separated by horizontal gel electrophoresis. Uninfected cells were used as a negative control (ni). The upper part demonstrates the VP60 specific mRNA of 1.7 kb in size. The lower part shows the 28S and 18S cellular rRNA, demonstrating that comparable amounts of RNA were loaded onto each gel lane.

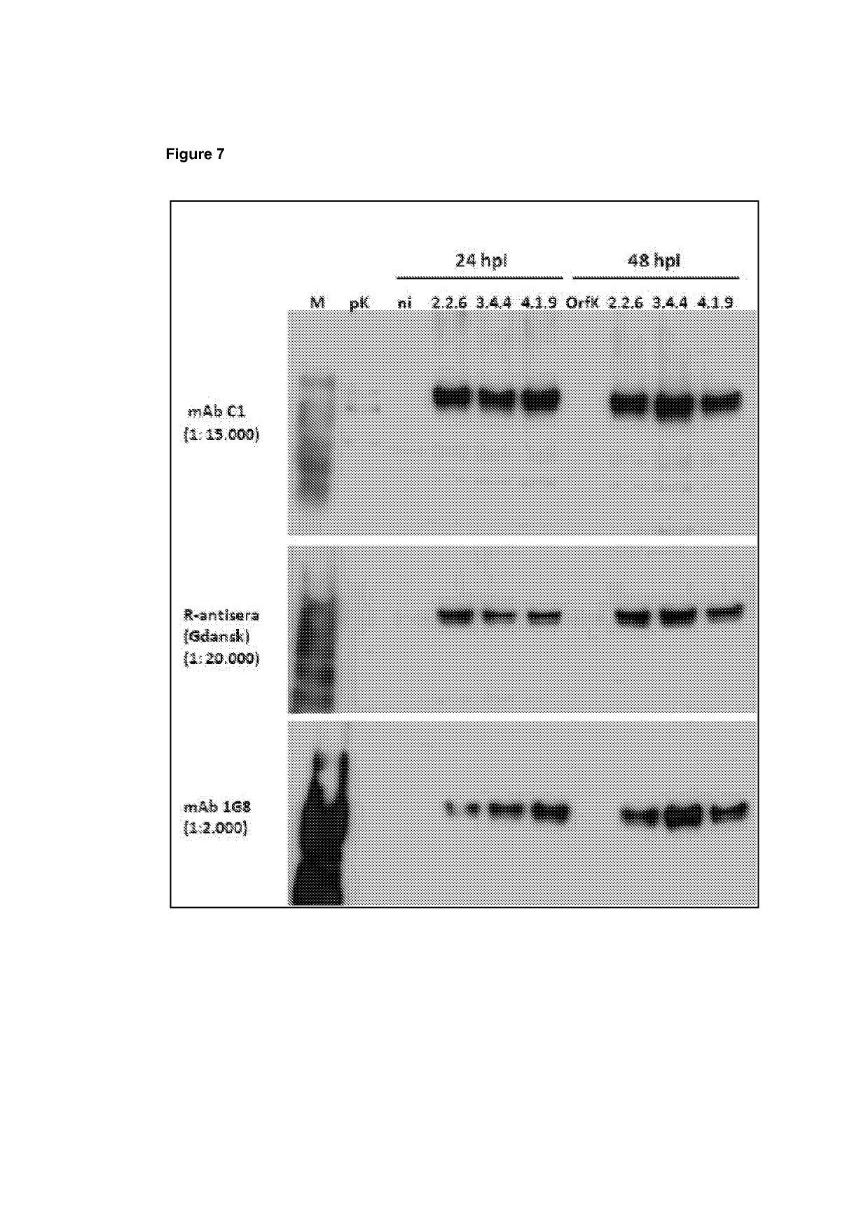

FIG. 7: Analysis of VP60 protein expression of 3 different recombinant plaque virus isolates by Western blot analysis. Lane OrfK contains lysate from cells infected with the parental virus D1701-VrV as control. Cell lysates were extracted 24 or 48 hours after infection and separated by standard PAGE. VP-60 was specifically detected with the monoclonal antibodies C1 and 1 G8 or the polyclonal rabbit antiserum (Gdansk).

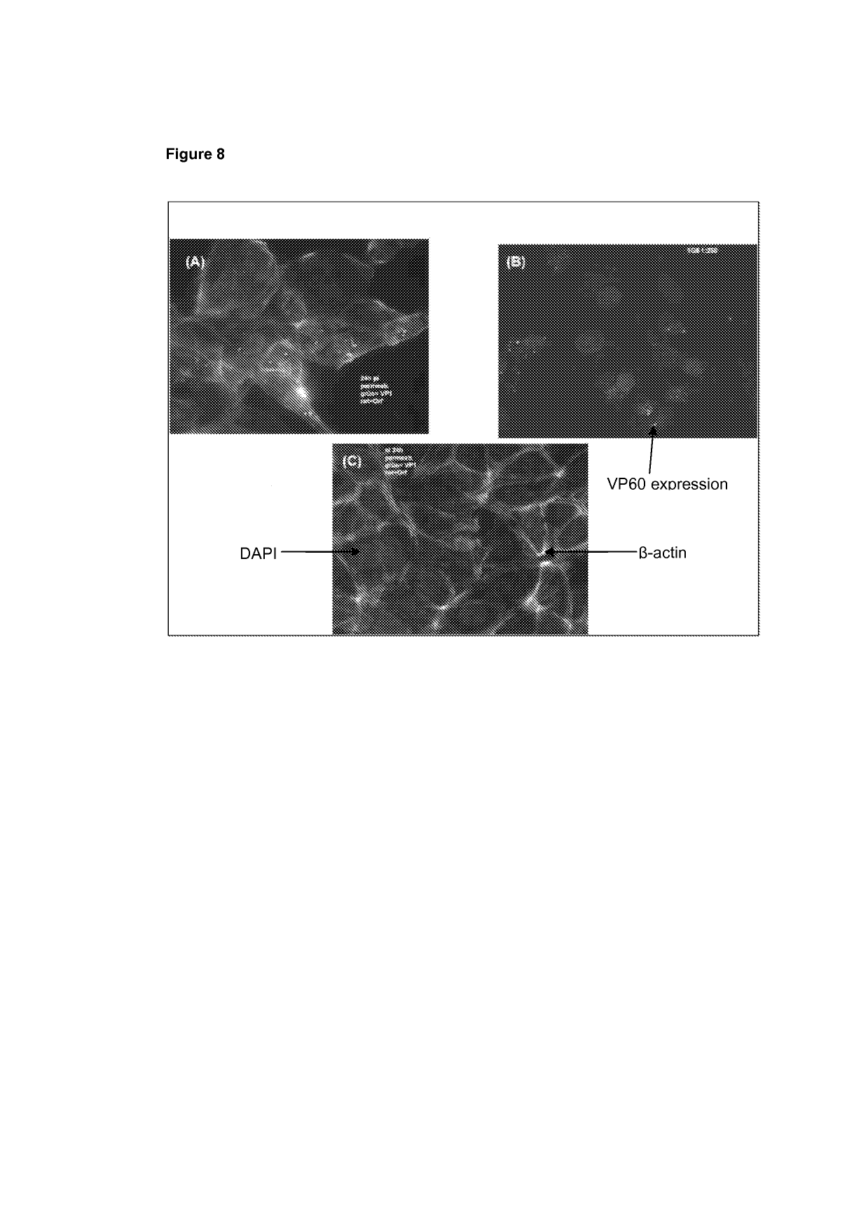

FIG. 8: Immunofluorescence analysis of VP60 expression (green staining, little dots, indicated by the arrow) using the VP60-specific mAB 1G8. Panels A and B represent cells infected for 24 hours with recombinant D1701-V-vp60n, panel C shows non-infected cells as control. Grey staining (bright staining, indicated by the arrow) depicts cellular β-actin, the cell nuclei are stained blue by DAPI (circular blurry staining, indicated by the arrow).

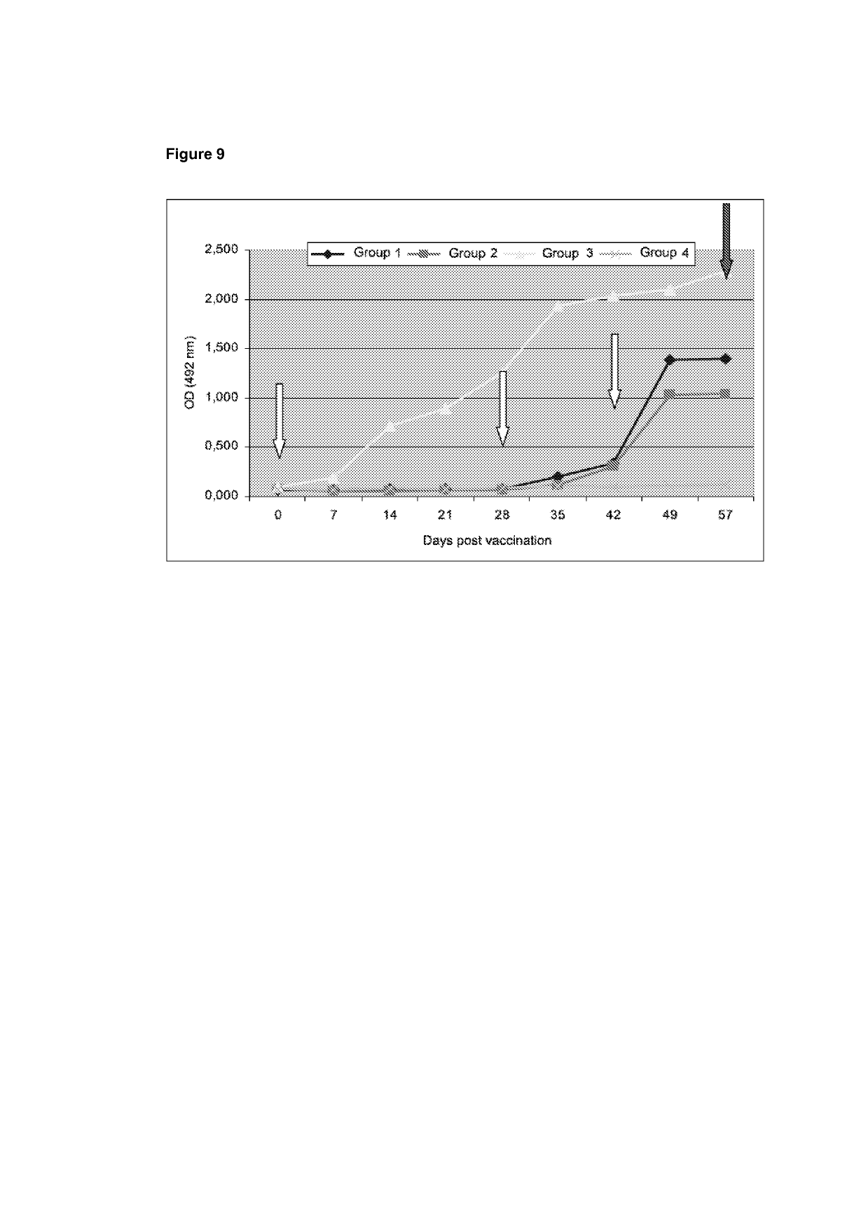

FIG. 9: Rabbit experiment investigating the immunogenicity of the recombinant D1701-V-vp60n. Results from sera tested by RHDV-antibody-ELISA test are shown for each animal group. Sera were weekly taken at the indicated days. The white arrows mark the time points of prime, first booster and second booster immunization, the red arrow denotes time of challenge infection with virulent RHDV. The antibody concentration is measured by OD (492 nm) determinations. Mean values were calculated from the data disclosed in table 1 across the 4 animals of each group.

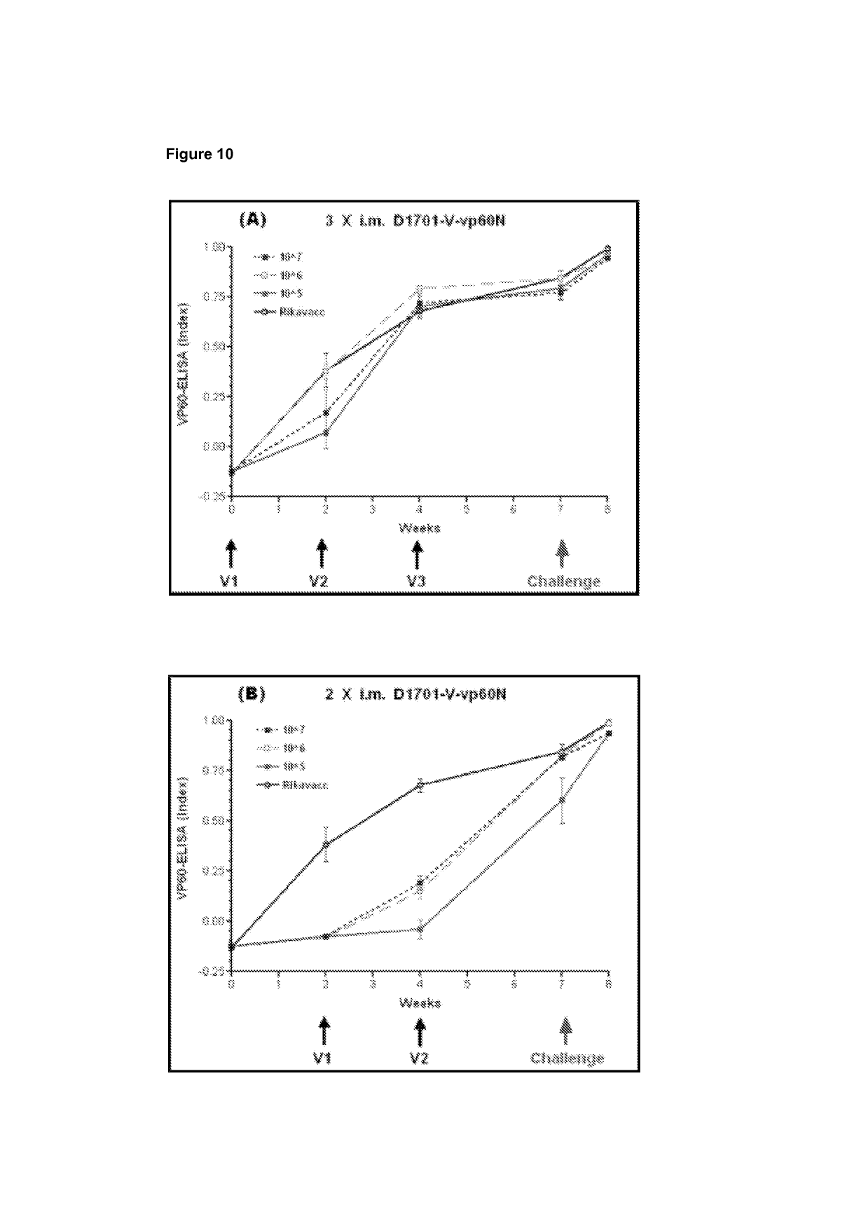

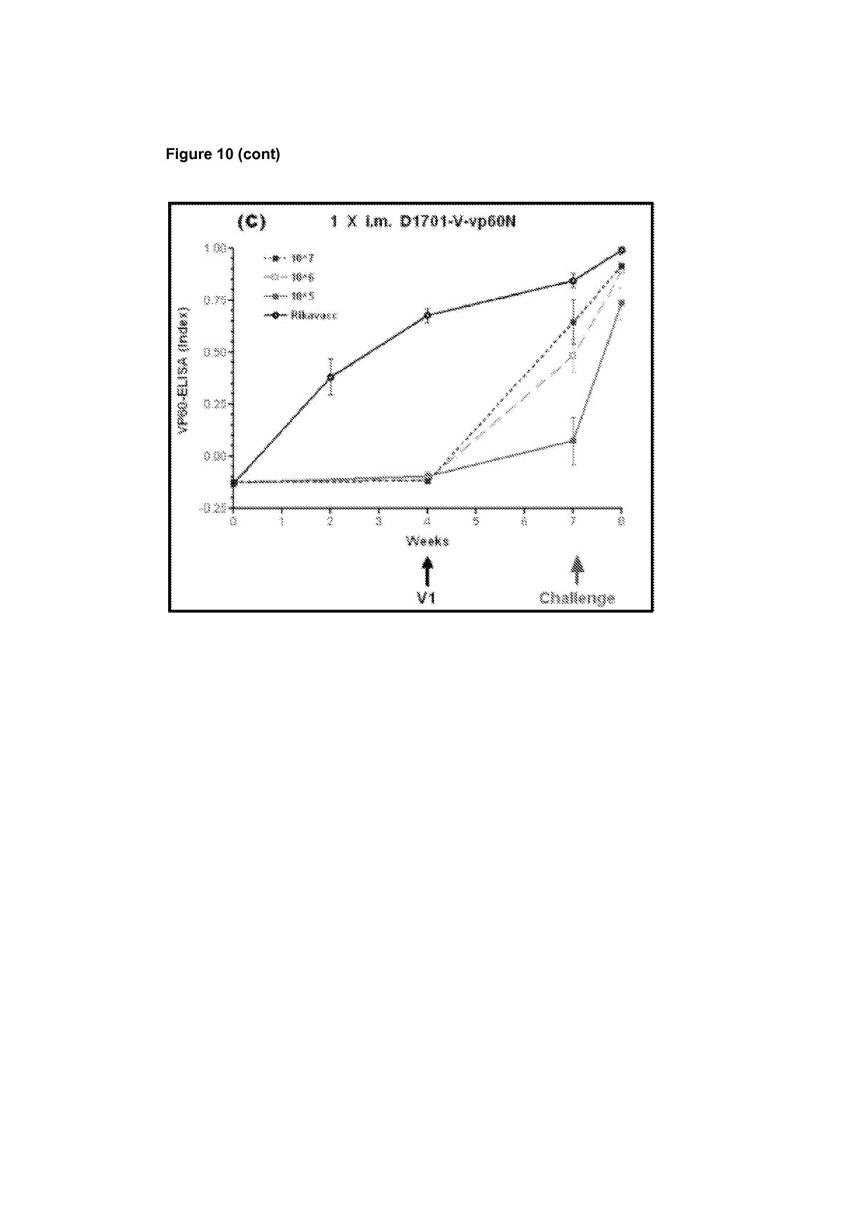

FIG. 10: Animal experiments investigating treatment dose. Serum antibody response (RHDV-ELISA) after immunisation with D1701-V-vp60n. Rabbits were treated intramuscularly as described once, twice or three times (V1, V2, V3) with the ORFV-recombinant. All animals were then challenge-infected 3 weeks after the last immunisation.

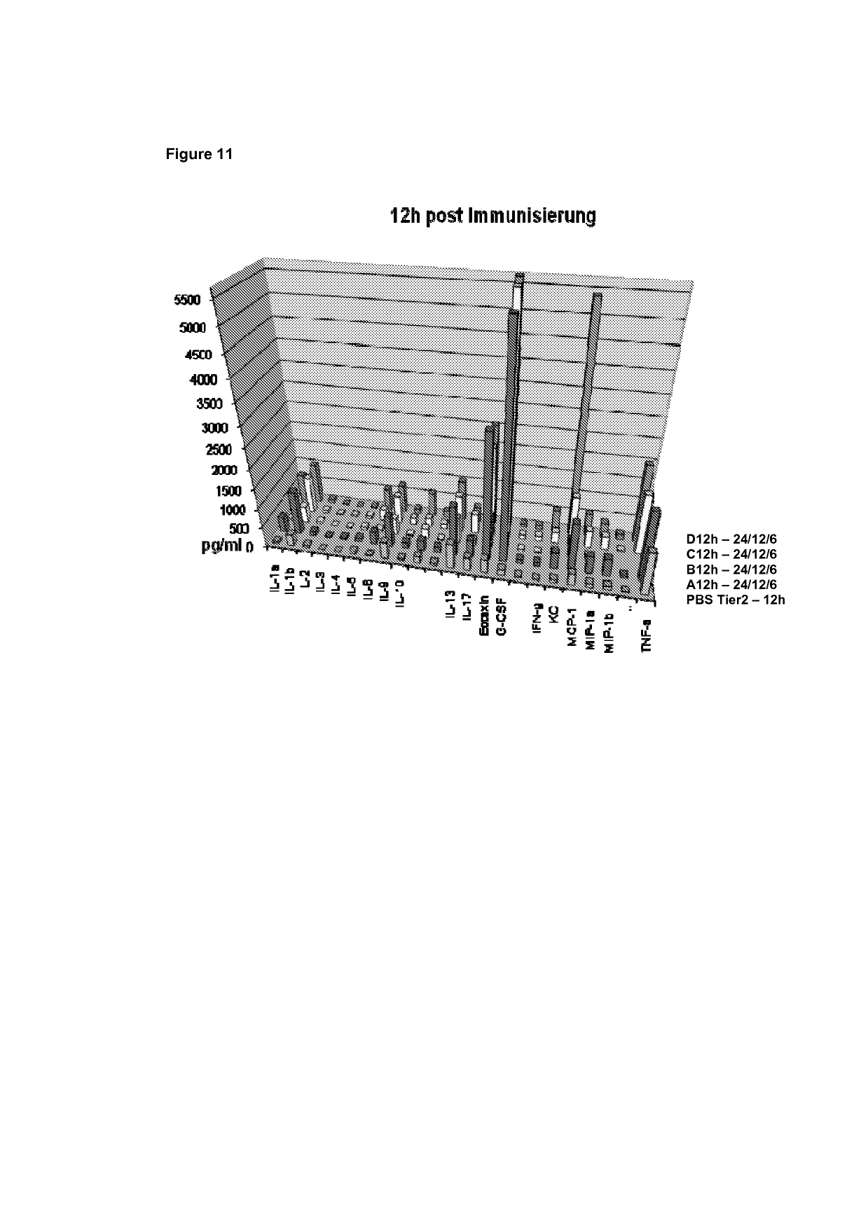

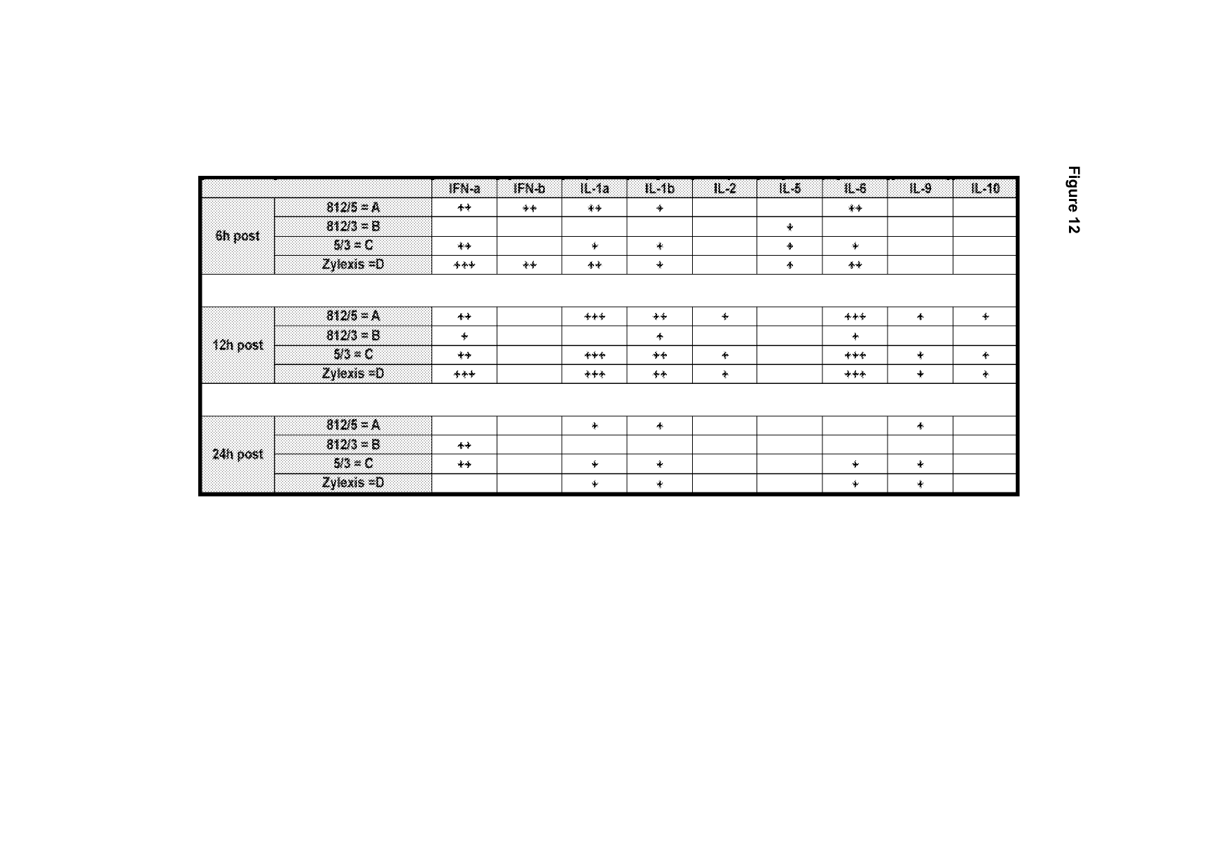

FIG. 11: Cytokine response in mouse serum after treatment with different viral preparations. 11-12 week-old Balb/C mice were treated intraperitoneally with 5×10⁶ PFU per animal. Animals used as negative controls were treated with PBS solution. Serum was prepared from the treated animals and the cytokine and chemokine expression was determined by multiplex ELISA. Results are shown 12 hours after prime immunisation.

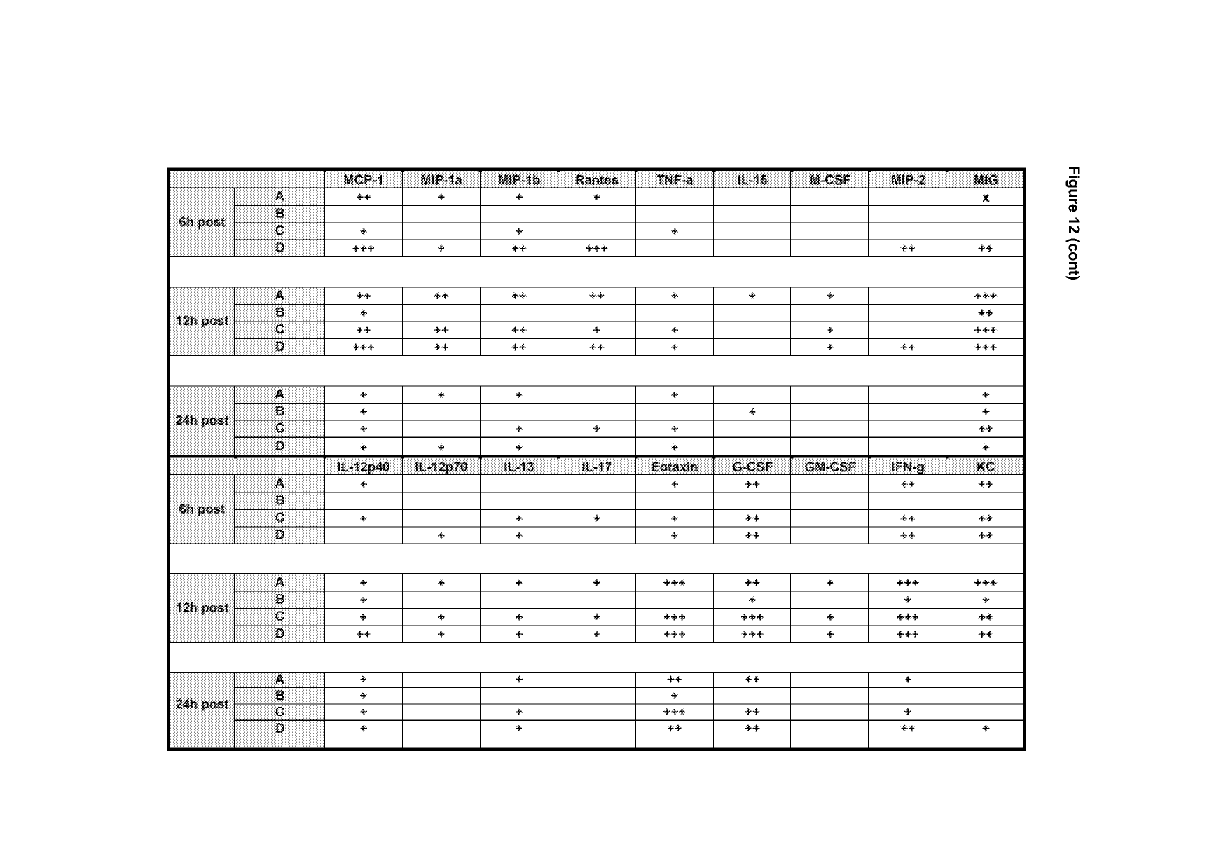

FIG. 12: Cytokines and Chemokines found in serum of Balb/C mice at the indicated hours after intraperitoneal application. Legend: +=up to 3-fold increase over PBS,++=more than 3-fold increase over PBS, +++=more than 6-fold increase over PBS, abbreviations: IFN: Interferon, IL: Interleukin, MCP: monocyte chemoattractant protein, MPI; macrophage inflammatory protein, MIG: monokine induced by interferon gamma, G-CSF: granulocyte colony stimulating factor, KC: keratinocytes chemoattractant.

### EXAMPLES

### Example 1

### VP60 DNA Sequence

In order to produce recombinant ORF viruses that express the VP60 protein of the RHDV (Rabbit Haemorrhagic Disease Virus), the entire VP60 gene (SEQ ID No. 1) was synthesised by the company “Mr. Gene” (Regensburg, Germany). In order to ensure correct expression of the recombinant VP60 the DNA sequence of the gene was optimised to remove early poxviral transcriptional stop motifs. The VP60 gene was provided in vector pMK-RQ, as shown in FIG. 1. The VP60 gene was isolated from the pMK-RQ plasmid by a HindIII-EcoR1 restriction digest and subsequently ligated into the transfer plasmid pDV-Rec1. The resulting plasmid was named pdV-VP60n. Correct insertion of the VP60 fragment was confirmed by DNA sequencing. See FIGS. 1 and 2 for a description of the VP60 DNA sequences.

### Example 2

### Recombinant Virus Selection Strategy

Vero cells were infected with the beta-galactosidase (LacZ-gene positive) expressing ORFV strain D1701-VrV and subsequently transfected with the pdV-VP60n plasmid via nucleofection. After successful DNA recombination, the beta-galactosidase gene is exchanged by the VP60 gene of RHDV to generate white recombinant virus plaques that can be distinguished from the parental blue D1701-VrV strains.

VP60 gene positive plaques can therefore be identified using plaque-PCR after selection according to colour (FIG. 3, vp60). After plaques had been purified three times, LacZ PCR-negative recombinant viruses could be obtained (FIG. 3, LacZ) that contained the desired VP60 gene. The three recombinant viruses marked by boxes in FIG. 3 (3.3.4, 4.1.9, 2.2.6) were selected for further testing.

### Example 3

### Immunostaining of Infected Cells for VP60 Expression

After propagation of the three plaque isolates, 3.3.4, 4.1.9 and 2.2.6, immunostaining of infected cells was carried out in order to test the expression of the VP60 protein. Either infected or uninfected cells were stained with a VP60-specific rabbit antiserum (FIG. 4, ni represents uninfected cells). In comparison to the uninfected cells (ni), all three isolates demonstrate comparably pronounced and specific staining of virus plaques.

### Example 4

### Integration of the VP60 Gene into the D1701-V Genome

Correct insertion of the VP60 gene into the genome of the ORFV D1701-V was analysed by restriction enzyme digestion and Southern blot hybridisation, using all three recombinants. The results are shown in FIG. 5. An internal VP60-specific probe was used for hybridisation. The size of the VP60 specific fragments are shown in FIG. 5. A schematic representation of the insertion site of the VP60 gene into the ORFV genome is provided in the lower panel.

The experimental results demonstrate correct insertion of the RHDV VP60 gene after exchange with the LacZ gene in all three plaque isolates of D1701-V-VP60. Further control hybridisations confirmed the correct gene insertion.

### Example 5

### Analysis of VP60 Transcription in Recombinant Viruses

RNA was isolated at various time points after infection (2 to 32 hours). VP60 specific RNA was detected using Northern blot hybridisation and a VP60-specific probe (FIG. 6, 1.7 kb, upper panel). VP60 transcription was detectable at the early phase of infection. Replication of the recombinant virus is not essential for VP60 gene expression, as demonstrated by the results obtained after cytosine arabinoside treatment (AraC: inhibition of viral DNA replication) or cyclohexamide treatment (CHX: only immediate early gene expression). The experimental results demonstrate that cells infected with recombinant virus show early expression of VP60 mRNA.

### Example 6

### Analysis of VP60 Protein Expression in Recombinant Viruses

Various specific antibodies were used to test protein expression of VP60 (60 kDa) by Western blot analysis (polyclonal rabbit anti-VP60 antiserum C1 from Prof. Dr. G.

Meyers, FLI Tübingen, polyclonal monospecific rabbit antiserum from Prof. B. Szewczyk, University of Gdansk, monoclonal antibody mAb1G8 from Dr. H. Schirrmeier, FLI Riems). Lysates were harvested at different time points (24 and 48 hours post infection). Lysates from uninfected cells were used as negative controls (ni) in addition to D1701-V infected cells. The cells were infected with an MOI of 1.0.

The results shown in FIG. 7 demonstrate a protein band at the correct molecular weight (ca. 60 kDa) using all 3 antibodies, clearly indicating that VP60 is correctly translated at both 24 and 48 hours post infection.

### Example 7

### Immunofluorescence Analysis

Immunofluorescence analysis using antibodies specific for the VP60 protein demonstrated globular-like VP60 expression in the cells infected with recombinant virus (FIG. 8). As can be seen in panels A and B, bright points of VP60 protein accumulate in the cytoplasm of infected cells.

### Example 8

### Animal Experiments Investigating the Immunogenicity of the ORFV Recombinant D1701-V-vp60n

Immunization experiments were carried out with 16 rabbits (chinchilla bastard) four months old and were sourced from the animal facility of the Friedrich-Loeffler-Institut (FLI), Island of Riems. The experimental design consisted of using four groups of animals, each group containing four rabbits.

Group 1 was treated with a prime immunisation intramuscularly (IM), comprising administration of recombinant virus of 1×10⁷ pfu/dosis (1 ml volume). Boosting treatments were subsequently carried out, the first boost occurring 28 days post vaccination, the second boost occurring 42 days post vaccination.

Group 2 was treated with a prime immunisation intramuscularly (IM), comprising administration of recombinant virus of 1×10⁶ pfu/dosis (1 ml volume), although otherwise carried out identically to the treatment of group 1.

Group 3 was treated with a prime immunisation using the control substance 1 ID RicaVacc. No boosting treatments were applied.

Group 4 was the control group, no treatment was applied.

Serial serum samples were obtained before immunisation in addition to 7, 14, 21, 28, 35, 42, 49, and 57 days past vaccination.

Challenge infection with virulent RHDV was carried out to test the immunogenicity of the administered recombinant virus. One ml of virulent RHDV of the “Eisenhüttenstadt” strain (10^{4.5} LD₅₀/ml) was used to inoculate the rabbits intramuscularly (IM) at 57 days post vaccination.

Diagnostic investigation was carried out using the following measurements:
1. Examination of the serum using an RHDV-antibody-ELISA test.
2. Clinical and pathomorphological examination of the animals after challenge.

Serology Results: Serum was extracted at weekly intervals over 57 days post vaccination up until challenge. Levels of antibodies directed against the VP60 antigen were measured using an RHDV-antibody-ELISA test according to standard protocols (FIG. 9). All RicaVacc-immunised animals demonstrated a sero-conversion as of the 14^{th} day post vaccination (group 3). The rabbits of groups 2 and 3 (ORF-RHDV recombinants) demonstrated a slight increase in antibody levels after the first boosting, in addition to a significant sero-conversion after the second boosting (third immunisation) (Table 2 and FIG. 8).

TABLE 2
Results from a serological examination using an
RHDV-antibody-ELISA test.
Days after infection

| | | | | | | | | | |
|---|---|---|---|---|---|---|---|---|---|
| # | 0 | 7 | 14 | 21 | 28 | 35 | 42 | 49 | 57 |

Group 1

| | | | | | | | | | |
|---|---|---|---|---|---|---|---|---|---|
| 381 | 0.049 | 0.084 | 0.091 | 0.082 | 0.087 | 0.194 | 0.235 | 1.007 | 0.982 |
| 380 | 0.078 | 0.056 | 0.057 | 0.061 | 0.063 | 0.244 | 0.265 | 0.965 | 0.758 |
| 371 | 0.079 | 0.067 | 0.069 | 0.068 | 0.067 | 0.202 | 0.49 | 1.484 | 1.684 |
| 370 | 0.064 | 0.063 | 0.058 | 0.079 | 0.079 | 0.174 | 0.355 | 2.103 | 2.17 |
| m | 0.068 | 0.068 | 0.069 | 0.073 | 0.074 | 0.204 | 0.336 | 1.39 | 1.399 |

Group 2

| | | | | | | | | | |
|---|---|---|---|---|---|---|---|---|---|
| 376 | 0.102 | 0.082 | 0.09 | 0.091 | 0.101 | 0.197 | 0.697 | 1.824 | 1.6 |
| 377 | 0.084 | 0.052 | 0.048 | 0.056 | 0.054 | 0.067 | 0.183 | 0.406 | 0.823 |
| 375 | 0.063 | 0.045 | 0.039 | 0.053 | 0.05 | 0.097 | 0.129 | 0.928 | 0.75 |
| 374 | 0.054 | 0.051 | 0.049 | 0.058 | 0.056 | 0.083 | 0.2 | 0.978 | 0.984 |
| m | 0.076 | 0.058 | 0.057 | 0.065 | 0.065 | 0.111 | 0.302 | 1.034 | 1.039 |

Group 3

| | | | | | | | | | |
|---|---|---|---|---|---|---|---|---|---|
| 369 | 0.065 | 0.234 | 0.872 | 0.437 | 0.67 | 1.395 | 1.712 | 1.933 | 2.152 |
| 368 | 0.102 | 0.157 | 0.582 | 1.37 | 1.441 | 2.066 | 2.274 | 2.244 | 2.406 |
| 367 | 0.065 | 0.231 | 0.508 | 0.558 | 1.09 | 1.951 | 1.735 | 1.82 | 2.063 |
| 366 | 0.133 | 0.153 | 0.905 | 1.215 | 1.837 | 2.339 | 2.428 | 2.422 | 2.556 |
| m | 0.091 | 0.194 | 0.717 | 0.895 | 1.26 | 1.938 | 2.037 | 2.105 | 2.294 |

Group 4

| | | | | | | | | | |
|---|---|---|---|---|---|---|---|---|---|
| 372 | 0.072 | 0.073 | 0.069 | 0.065 | 0.085 | 0.089 | 0.089 | 0.083 | 0.096 |
| 373 | 0.056 | 0.048 | 0.06 | 0.052 | 0.063 | 0.079 | 0.061 | 0.05 | 0.048 |
| 379 | 0.083 | 0.064 | 0.11 | 0.084 | 0.1 | 0.098 | 0.102 | 0.088 | 0.09 |
| 378 | 0.065 | 0.065 | 0.086 | 0.08 | 0.088 | 0.146 | 0.128 | 0.182 | 0.236 |
| m | 0.069 | 0.063 | 0.081 | 0.07 | 0.084 | 0.103 | 0.095 | 0.101 | 0.118 |

Challenge results: Four animals of group 4 (controls) died within 48 hours after infection with virulent RHDV. All animals of groups 1, 2, and 3 survived the challenge infection. None of the immunised animals of groups 1, 2 or 3 demonstrated any clinical symptoms of RHDV infection. The pathomorphological analysis of the perished animals of group 4 revealed symptoms associated with infection with RHDV (haemorrhagic septicaemia and liver damage).

These results demonstrate that the recombinant virus of the present invention is capable of stimulating the production of anti-VP60 antibodies, and more importantly to mediate protection against lethal RHDV infection.

It was also demonstrated that the animals could be protected by vaccination with D1701-V-VP60n in the absence of detectable anti-VP60 serum antibodies.

### Example 9

### Animal Experiments Investigating Treatment Dose

The second set of animal experiments was designed to test the number of immunisations and the vaccination dose of D1701-V-vp60n, which is necessary to provide effective protection in rabbits against a challenge infection. Rabbits (n=4) were immunised once, twice or three times intramuscularly with 10⁷, 10⁶ or 10⁵ PFU of the ORFV-recombinants before challenge infection with virulent RHDV (FIG. 10).

The results demonstrate that all D1701-V-vp60n-immunised animals were completely protected against RHDV infection. It is also shown that in fact booster-immunisations with ORFV-recombinants are required for clear proof of specific antibodies in serum (FIG. 10).

The animal experiments described herein provide evidence that a single intramuscular immunization with the lowest tested dose of ORFV-recombinants D1701-V-VP60n (10⁵ PFU) provides effective protection against a challenge infection with virulent RHDV. The protective effect was comparable to an immunization with the commercially available RIKA-VACC® RHD, which is obtained from RHDV-infected liver material after inactivation. These examples therefore demonstrate the protective working of the recombinant VP60-expressing recombinant viruses as claimed.

### Example 10

### Immune Stimulatory Effects of D1701

In order to test the immune-modulating properties of virus preparations, 11-12 week old Balb/C mice were treated with 5×10⁶ PFU per animal in an intraperitoneal immunisation. Animals used as negative controls were treated with PBS solution.

After 6, 12 or 24 hours blood from the treated animals was isolated, serum was extracted and the cytokine and chemokine expression was tested using ELISA kits. Furthermore, the spleens of each of the tested animals were isolated for testing the activation of NK (natural killer) cells in a lymphocyte-proliferation test.

34 cytokines and chemokines were measured using a multiplex ELISA system (BioRad, Germany). The results of this analysis are summarized in FIGS. 11 and 12.

The results demonstrated that 12 hours after immunisation the maximum expression of the measured cytokines appeared and decreased after 24 hours. Groups A, B and C represent different preparations of D1701 virus, whereby group D represents a vaccine from the prior art. Group C comprised a mixture of two different viral preparations.

As is shown in FIG. 12, immunisation with A, C and D led to an inflammatory immune response, which is defined by cytokine expression IL-1a, IL-1b, IL-6 and G-CSF. An enhanced expression of type 1 interferon, especially IFN-alpha, was also observed. These results demonstrate an immune stimulatory effect mediated by the applied ORFV preparations.

The activity of NK-cells isolated from the spleen was also tested. The NK-cells were tested using both the chromium-release assay and by measuring cell proliferation of the splenocytes. The chromium-release assay demonstrated that 12 hours after application of the viral preparations a clear increase in the cytolytic capacity of the NK-cells of the groups C and D can be observed. 24 hours after immunisation the cytolitic activity was further increased.

In order to test splenocyte proliferation a cell proliferation test using a BrdU-ELISA test was applied. The splenocytes were cultured at 37° C. and 5% CO₂ for three days before cell proliferation was stimulated. 6 hours after immunisation there was no significant increase in cell proliferation. However, after 12 hours the groups A and C demonstrated a clearly increased level of proliferation.

Together these immune stimulatory tests demonstrate that between 12 and 24 hours after immunisation activation of the innate immunity of the treated animals was observed. These results further demonstrate that the parapox viral vectors of the present invention provide an adjuvant effect when used as a vaccine.

1. A recombinant parapoxvirus comprising VP60 major capsid protein from the rabbit haemorrhagic disease virus (RHDV).

2. The parapoxvirus according to claim 1, wherein the VP60 major capsid protein from the rabbit haemorrhagic disease virus (RHDV) is expressed from a foreign nucleic acid sequence.

3. The parapoxvirus according to claim 1, wherein the parapoxvirus is an Orf virus (ORFV), preferably the ORFV-strain D1701, more preferably D1701-V-VP60n.

4. The parapoxvirus according to claim 42, wherein the foreign nucleic acid sequence is a DNA sequence integrated into the parapoxvirus genome.

5. The parapoxvirus according to claim 1, wherein the VP60 major capsid protein is encoded by a DNA sequence comprising the sequence according to SEQ ID No. 1 or SEQ ID No. 2.

6. The parapoxvirus according to claim 1, wherein the VP60 major capsid protein comprises the amino acid sequence according to SEQ ID No. 3.

7-8. (canceled)

9. A composition comprising the parapoxvirus according to claim 1, a nucleic acid molecule encoding the VP60 protein according to SEQ ID No. 1 or SEQ ID No. 2 and/or the VP60 protein comprising the sequence according to SEQ ID No. 3 and a pharmaceutically acceptable carrier.

10. (canceled)

11. Vaccine comprising the parapoxvirus according to claim 1, whereby the parapoxvirus is preferably alive.

12. The vaccine according to the preceding claim 11, wherein that additional agents are present for enhanced vaccine function, such as adjuvants or immune stimulators.

13. The vaccine according to claim 11, wherein that the vaccine is a combination vaccine, comprising of vaccine-agents for additional diseases.

14. A method of therapeutic and/or prophylactic treatment of RHDV infection in rabbits, wherein that a therapeutically effective amount of the parapoxvirus according to claim 1, is administered to a subject.

15. An isolated nucleic acid molecule encoding a rabbit haemorrhagic disease virus (RHDV) protein VP60 comprising the sequence according to SEQ ID No. 1 or SEQ ID No. 2.

16. (canceled)

17. VP60 major capsid protein of the rabbit haemorrhagic disease virus (RHDV) comprising the sequence according to SEQ ID No. 3.

18. (canceled)


SEQUENCE LISTING (3 sequences)

SEQ ID NO: 1 - DNA, 1740 bp, Unknown
  VP60 Gene sequence
atggagggcaaagcccgtgcagcgccgcaaggcgaagcagcgggcactgccaccacagcatcagttcccggaaccacgactgatggcatggatcctggcgttgtggccactaccagcgtgatcactgcagaaaattcatccgcatcgattgcaacagcagggattggcggaccaccccaacaggtggaccaacaagagacatggagaacaaatttttattacaatgatgttttcacttggtcagtcgcggacgcccctggcagcatactttacaccgtccaacattctccacaaaacaacccattcacagccgtgctgagccagatgtacgctggctgggctggtggcatgcagtttcgcttcatagttgccgggtcaggtgtgtttggtgggcgactggtcgcggctgtgataccaccaggcatcgagattggaccagggttggaggttaggcagtttcctcatgttgtcatcgatgcccgttcactcgaacctgttaccatcaccatgccagacttgcgtcccaacatgtaccatccaactggtgaccctggccttgttcccacactagtccttagtgtttacaacaaccttatcaacccgtttggtgggtccactagtgcaatccaggtgacagtggaaacaaggccaagtgaagactttgagttcgtgatgattcgagccccctccagcaagactgttgactcaatttcgcccgcaggcctcctcacgaccccagtccttactggggttggcaatgacaacaggtggaacggccaaatagtgggactgcaaccagtacctggagggttttctacgtgcaacaggcattggaacttgaatggcagcacatatggctggtcaagccctcggtttgccgacattgaccatcgaagaggcagtgcaagttactctgggagcaacgcaaccaacgtgcttcagttttggtatgccaatgctgggtctgcaatcgacaaccccatttcccaggttgcaccagacggctttcctgacatgtcgttcgtgccctttaacggccccggcattccagctgcggggtgggttggatttggtgcaatctggaacagtaacagcggtgcccccaacgtcacgactgtgcaggcctatgagttaggttttgccactggggcaccaggcaacctccagtccaccaccaacacttcaggtgcacagactgtcgccaagtccatttatgccgtggtgactggcacagcccaaaacccagccggattgtttgtgatggcctcgggtattatctccaccccaaatgccagcgccatcacatacacgcctcaaccagacagaattgtaaccacacccggcactcctgccgctgcacctgtgggtaagaacacacccatcatgtttgcgtctgtcgtcaggcgcaccggtgacgtcaacgccacagctgggtcagccaacgggacccagtacggcacaggctcccaaccactgccagtgacaattggactttcgctcaacaactactcgtcagcacttatgcccggacagtttttcgtttggcagttaacctttgcatctggtttcatggagataggtttaagtgtggacgggtatttttatgcaggaacaggggcctcaaccacactcattgacctgactgaactcattgacgtacgccctgtgggacccaggccgtccaaaagcacactcgtgttcaacctggggggcacagccaatggcttttcttatgtctga

SEQ ID NO: 2 - DNA, 1768 bp, Unknown
  VP60 gene sequence (optimised; VP60-new)
gtgaagcttatggagggcaaagcccgtgcagcgccgcaaggcgaagcagcgggcactgccaccacagcatcagttcccggaaccacgactgatggcatggatcctggcgttgtggccactaccagcgtgatcactgcagaaaattcatccgcatcgattgcaacagcagggattggcggaccaccccaacaggtggaccaacaagagacatggagaacaaatttctactacaatgatgttttcacttggtcagtcgcggacgcccctggcagcatactttacaccgtccaacattctccacaaaacaacccattcacagccgtgctgagccagatgtacgctggctgggctggtggcatgcagtttcgcttcatagttgccgggtcaggtgtgtttggtgggcgactggtcgcggctgtgataccaccaggcatcgagattggaccagggttggaggttaggcagtttcctcatgttgtcatcgatgcccgttcactcgaacctgttaccatcaccatgccagacttgcgtcccaacatgtaccatccaactggtgaccctggccttgttcccacactagtccttagtgtttacaacaaccttatcaacccgtttggtgggtccactagtgcaatccaggtgacagtggaaacaaggccaagtgaagactttgagttcgtgatgattcgagccccctccagcaagactgttgactcaatttcgcccgcaggcctcctcacgaccccagtccttactggggttggcaatgacaacaggtggaacggccaaatagtgggactgcaaccagtacctggagggttttctacgtgcaacaggcattggaacttgaatggcagcacatatggctggtcaagccctcggtttgccgacattgaccatcgaagaggcagtgcaagttactctgggagcaacgcaaccaacgtgcttcagttttggtatgccaatgctgggtctgcaatcgacaaccccatttcccaggttgcaccagacggctttcctgacatgtcgttcgtgccctttaacggccccggcattccagctgcggggtgggttggatttggtgcaatctggaacagtaacagcggtgcccccaacgtcacgactgtgcaggcctatgagttaggttttgccactggggcaccaggcaacctccagtccaccaccaacacttcaggtgcacagactgtcgccaagtccatttatgccgtggtgactggcacagcccaaaacccagccggattgtttgtgatggcctcgggtattatctccaccccaaatgccagcgccatcacatacacgcctcaaccagacagaattgtaaccacacccggcactcctgccgctgcacctgtgggtaagaacacacccatcatgtttgcgtctgtcgtcaggcgcaccggtgacgtcaacgccacagctgggtcagccaacgggacccagtacggcacaggctcccaaccactgccagtgacaattggactttcgctcaacaactactcgtcagcacttatgcccggacagttcttcgtttggcagttaacctttgcatctggtttcatggagataggtttaagtgtggacgggtatttctacgcaggaacaggggcctcaaccacactcattgacctgactgaactcattgacgtacgccctgtgggacccaggccgtccaaaagcacactcgtgttcaacctggggggcacagccaatggcttttcttatgtctgatgatttttatgaattcatc

SEQ ID NO: 3 - protein, 579 aa, Unknown
  VP60 Protein sequence
MEGKARAAPQGEAAGTATTASVPGTTTDGMDPGVVATTSVITAENSSASIATAGIGGPPQQVDQQETWRTNFYYNDVFTWSVADAPGSILYTVQHSPQNNPFTAVLSQMYAGWAGGMQFRFIVAGSGVFGGRLVAAVIPPGIEIGPGLEVRQFPHVVIDARSLEPVTITMPDLRPNMYHPTGDPGLVPTLVLSVYNNLINPFGGSTSAIQVTVETRPSEDFEFVMIRAPSSKTVDSISPAGLLTTPVLTGVGNDNRWNGQIVGLQPVPGGFSTCNRHWNLNGSTYGWSSPRFADIDHRRGSASYSGSNATNVLQFWYANAGSAIDNPISQVAPDGFPDMSFVPFNGPGIPAAGWVGFGAIWNSNSGAPNVTTVQAYELGFATGAPGNLQSTTNTSGAQTVAKSIYAVVTGTAQNPAGLFVMASGIISTPNASAITYTPQPDRIVTTPGTPAAAPVGKNTPIMFASVVRRTGDVNATAGSANGTQYGTGSQPLPVTIGLSLNNYSSALMPGQFFVWQLTFASGFMEIGLSVDGYFYAGTGASTTLIDLTELIDVRPVGPRPSKSTLVFNLGGTANGFSYV